(19) United States
(12) Patent Application Publication
(10) Pub. No.: US 2010/0209927 A1
(43) Pub. Date: Aug. 19, 2010

(54) PROCESSING DEVICE TABLET

(76) Inventors: Vinod P. Menon, Woodbury, MN (US); Ranjani V. Parthasarathy, Woodbury, MN (US)

Correspondence Address: 3M INNOVATIVE PROPERTIES COMPANY, PO BOX 33427, ST. PAUL, MN, 55133-3427, US

(21) Appl. No.: 12/681,898

(22) PCT Filed: Nov. 6, 2008
(86) PCT No.: PCT/US08/82543

§ 371 Date: Apr. 7, 2010

(30) Foreign Application Priority Data
Nov. 6, 2007 (US) 60985933
Nov. 6, 2007 (US) 60985941

(51) Int. Cl. C12Q 1/68; G01N 33/00; C12M 1/34

(52) U.S. Cl. 435/6; 436/89; 422/68.1; 435/287.2

(57) ABSTRACT

A microfluidic processing device includes a tablet comprising a reagent, where the tablet is configured to fit within at least one chamber of the processing device. In addition, in some embodiments, at least two tablets are disposed within a single process chamber of the processing device. Further, in some embodiments, each tablet may comprise one or more different types of reagents. In some embodiments, the tablet is a microtablet including a greatest dimension of less than about five millimeters.

### CROSS REFERENCE TO RELATED APPLICATIONS

This application claims the benefit of U.S. Provisional Patent Application Ser. Nos. 60/985,941, filed Nov. 6, 2007 and 60/985,933, filed Nov. 6, 2007, both of which are incorporated herein by reference.

### TECHNICAL FIELD

The invention relates to a processing device, and, more particularly, a processing device including a reagent.

### BACKGROUND

In some processing techniques, such as processing techniques that require different chemical, biochemical, and other reactions that are sensitive to temperature variations, it may be desirable to process samples with a processing device including multiple chambers in which different portions of one sample or different samples can be processed simultaneously. Although it may be possible to process samples individually and obtain accurate sample-to-sample results, individual processing can be relatively time-consuming and expensive.

One type of processing device is a microfluidics-based analytical device, which may also be referred to as a “microfluidic processing device.” A microfluidic device can offer unique advantages in sample handling, reagent mixing, separation, and detection. Additionally, the use of microfluidic devices generally allows for relatively low fabrication cost, enhancement of analytical performance, relatively low power budget, and low consumption of chemicals when compared to conventional fluidic systems.

### SUMMARY

In general, the invention is related to methods for manufacturing a regent in tablet form for use in a processing device, particularly a microfluidic sample processing device. Moreover, the invention is related to methods and assemblies for using a reagent in tablet form, such as, for example, with a microfluidic sampling device.

In one embodiment, the invention is directed to a method comprising selecting at least one reagent and forming a tablet comprising the at least one reagent and at least one matrix material. The tablet dimensions are configured to fit within at least one chamber of a microfluidic processing device.

In another embodiment, the invention is directed to a method comprising introducing a sample into a microfluidic processing device and at least partially dissolving a tablet in a chamber of the microfluidic device. The tablet comprises a reagent and a matrix material and is configured to fit within the chamber of the microfluidic processing device.

In another embodiment, the invention is directed to an assembly comprising a microfluidic processing device comprising an input chamber and a process chamber fluidically coupled to the sample input chamber; and a tablet comprising a reagent and a matrix material. The tablet is configured to fit within the process chamber of the microfluidic processing device.

In another embodiment, the invention is directed to a method comprising selecting an active component, selecting a substantially solid reconstitution buffer, and forming a tablet comprising the active component and the substantially solid reconstitution buffer. In some embodiments, the method further comprises selecting a matrix material and forming the tablet comprising the matrix material. In some embodiments, the active component comprises an enzyme. The tablet may be sized to fit within at least one chamber of a microfluidic processing device.

In another embodiment, the invention is directed to an assembly comprising a microfluidic processing device comprising an input chamber and a process chamber fluidically coupled to the input chamber. The assembly further comprises a tablet comprising an active component and a substantially solid reconstitution buffer, wherein the tablet is configured to fit within the process chamber of the microfluidic processing device.

In another embodiment, the invention is directed to a method comprising introducing an analyte into a microfluidic sample processing device, and at least partially dissolving a tablet in a chamber of the microfluidic device. The tablet comprises an active component and a substantially solid reconstitution buffer, and is configured to fit within the chamber of the microfluidic processing device.

Embodiments of the present invention may provide for one or more advantages. For example, some embodiments include a method of forming a reagent in tablet form that may allow for ease in handling and introduction of a reagent to a sample processing device, and allow for a portable reagent form for use in processing devices. Generally, reagents in tablet form may allow for high throughput manufacture of reagent doses for use in processing devices by an established process, high throughput assembly of processing devices including reagents, and also may allow for high throughput processing in such processing devices. A high throughput assembly may be achieved because a dry reagent may be introduced into a sample processing device without requiring a drying time for a reagent in the processing device. Additionally, some embodiments may allow for a large amount of reagent to be introduced into a sample processing device relative to the total volume of a tablet introduced into the device.

In another example, some embodiments may allow for precision introduction or spotting of one reagent or multiple reagents in a single chamber of a processing device, and also contain a reagent within the boundaries of such a chamber. Moreover, a tablet may provide high content uniformity of tablet components, including reagent components, throughout the volume of the tablet. Furthermore, a reagent in tablet form may exhibit excellent mechanical stability, e.g. dimensional stability, which may aid assembly of the reagent tablet into the processing device.

In another example, embodiments of the present invention may allow for compression of at least a reagent to form a tablet without significant deleterious results to the reagent, e.g., no denaturation to an enzyme reagent. In another example, embodiments of a tablet manufacturing process may prevent tablet components from being exposed to water during the manufacturing process. Additionally, some embodiments may provide for a tablet containing a reagent to adequately dissolve at an acceptable rate, including embodiments in which the tablet may be considered a microtablet. Certain embodiments may also allow for microtablets with very high active dose incorporation even with components that are poorly compressible in macroscopic form.

In another example, some embodiments may allow for a tablet to include either insoluble or soluble components, while still some embodiments may include both soluble and insoluble components. For example, soluble components such as inert disintegrants can be added to aid in the dissolution process. The disintegrants may aid dissolution of the tablet, e.g., in combination with rotating or otherwise agitating the processing device.

### SUMMARY OF EXEMPLARY EMBODIMENTS

A representative listing of some of the possible exemplary embodiments follows:

1. A method comprising selecting at least one reagent; and forming a tablet comprising the at least one reagent and at least one matrix material, wherein the tablet is sized to fit within at least one chamber of a microfluidic processing device.

2. The method of embodiment 1, wherein forming the tablet comprises compressing the at least one reagent and the at least one matrix material to define the tablet.

3. The method of embodiment 2, wherein compressing the at least one reagent and the at least one matrix material comprises compressing the at least one reagent and the at least one matrix material the tablet via a tablet press.

4. The method of any of embodiments 2-3, wherein compressing the at least one reagent and the at least one matrix material comprises compressing the at least one reagent and the at least one matrix material at a pressure in a range of about 15 megapascals to about 200 megapascals.

5. The method of any of embodiments 1-4, wherein the tablet further comprises a lubricant material.

6. The method of any of embodiments 1-5, wherein forming the tablet comprises forming the tablet comprising a substantially uniform distribution of the at least one reagent and the at least one matrix material.

7. The method of any of embodiments 1-6, further comprising lyophilizing the at least one reagent and the at least one matrix material prior to forming the tablet.

8. The method of any of embodiments 1-7, wherein the at least one matrix material comprises an insoluble material, the method further comprising spraying the at least one reagent onto the insoluble material and dehydrating the insoluble material prior to forming the tablet.

9. The method of any of embodiments 1-8, further comprising dry mixing the at least one reagent and the at least one matrix material prior to forming the tablet.

10. The method of any of embodiments 1-9, wherein the at least one reagent comprises a first reagent, the method further comprising selecting a second reagent, and forming the tablet comprises forming the tablet comprising the first reagent, the second reagent, and the at least one matrix material.

11. The method of any of embodiments 1-9, wherein the at least one reagent comprises a first reagent and the tablet comprises a first tablet, the method further comprising selecting a second reagent; and forming a second tablet comprising at least the second reagent, wherein the second tablet is sized to fit within at least one chamber of the microfluidic sample processing device.

12. The method of any of embodiments 1-11, where the at least one reagent comprises lysostaphin.

13. The method of any of embodiments 1-12, wherein forming the tablet comprises forming the tablet in an environment comprising a relative humidity of about 1% to about 30%.

14. The method of any of embodiments 1-13, wherein the tablet is a microtablet with a greatest dimension in a range of about 0.5 millimeters to about 5 millimeters.

15. A method comprising introducing an analyte into a microfluidic sample processing device; and at least partially dissolving a tablet in a chamber of the microfluidic device, wherein the tablet comprises a reagent and a matrix material and is configured to fit within the chamber of the microfluidic processing device.

16. The method of embodiment 15, wherein the matrix material comprises a solubility of about 0 grams per 100 grams of water to about 400 grams per 100 grams of water.

17. The method of any of embodiments 15-16, wherein the tablet substantially dissolves in the chamber within about 30 seconds to about 300 seconds from an introduction of a fluid into the chamber.

18. The method of embodiment 17, wherein the tablet substantially dissolves in the chamber within about 30 seconds to about 180 seconds from an introduction of a fluid into the chamber.

19. The method of any of embodiments 15-18, wherein the tablet comprises a first tablet, the method further comprising at least partially dissolving a second tablet in the chamber of the microfluidic device, the second tablet comprising a second reagent.

20. The method of embodiment 19, wherein the second tablet further comprises a second matrix material.

21. The method of embodiment 20, wherein the first matrix material exhibits a higher solubility in water than the second matrix material.

22. The method of any of embodiments 19-21, wherein the first tablet dissolves in the chamber at a greater rate than the second tablet.

23. The method of any of embodiments 15-22, wherein the reagent comprises a first reagent, the first tablet further comprising a second reagent different than the first reagent.

24. The method of any of embodiments 15-23, further comprising processing the analyte with the reagent, wherein processing the sample comprises at least one of preparing the sample, nucleic acid amplification, detecting or assaying a nucleic acid or detecting or assaying an amino acid.

25. The method of any of embodiments 1-24, wherein the reagent comprises at least one of a lysis reagent, a protein-digesting reagent, a nucleic acid amplifying enzyme, an oligonucleotide, a probe, nucleotide triphosphates, a buffer, a salt, a surfactant, a dye, a nucleic acid control, a reducing agent, dimethyl sulfoxide (DMSO), ethylenediaminetetraacetic acid (EDTA), ethylene glycol-bis(2-aminoethylether)-N,N,N′,N′-tetraacetic acid (EGTA), microspheres capable of binding a nucleic acid or a combination thereof.

26. The method of any of embodiments 1-25, wherein the matrix material comprises at least one of a water soluble polymer, a carbohydrate and a combination thereof.

27. The method of embodiment 26, wherein the carbohydrate is selected from the group consisting of sucrose, dextran, trehalose, pullulan, α-cyclodextrin, mannitol, sorbitol, and a combination thereof.

28. The method of any of embodiments 1-27, wherein the tablet includes about 1 percent to about 95 percent by tablet weight of the reagent.

29. An assembly comprising: a microfluidic processing device comprising an input chamber; and a process chamber fluidically coupled to the input chamber; and a tablet comprising a reagent and a matrix material, wherein the tablet is configured to fit within the process chamber of the microfluidic processing device.

30. The assembly of embodiment 29, wherein the tablet is sealed within the process chamber of the microfluidic processing device.

31. The assembly of any of embodiments 29-30, wherein the matrix material comprises a solubility of about 0 grams per 100 grams of water to about 400 grams per 100 grams of water

32. The assembly of any of embodiments 29-31, wherein the tablet substantially dissolves in the chamber within about 30 seconds to about 300 seconds from an introduction of a fluid into the chamber.

33. The assembly of embodiment 32, wherein the tablet substantially dissolves in the chamber within about 30 seconds to about 180 seconds from an introduction of a fluid into the chamber.

34. The assembly of any of embodiments 29-33, wherein the tablet comprises a first tablet, the assembly further comprising a second tablet in the chamber of the microfluidic device, the second tablet comprising a second reagent.

35. The assembly of embodiment 34, wherein the second tablet further comprises a second matrix material.

36. The assembly of embodiment 35, wherein the first matrix material exhibits a higher solubility in water than the second matrix material.

37. The assembly of any of embodiments 34-36, wherein the first tablet dissolves in the chamber at a greater rate than the second tablet.

38. The assembly of any of embodiments 29-37, wherein the reagent comprises a first reagent, the first tablet further comprising a second reagent different than the first reagent.

39. The assembly of any of embodiments 29-38, wherein the at least one reagent is used in at least one of a step of sample preparation, a step of nucleic acid amplification, a step of detection in a process for detecting or assaying a nucleic acid, or a step of detection in a process for detecting or assaying a amino acid.

40. The assembly of any of embodiments 29-39, wherein the at least one reagent comprises at least one of a lysis reagent, a protein-digesting reagent, a nucleic acid amplifying enzyme, an oligonucleotide, a probe, nucleotide triphosphates, a buffer, a salt, a surfactant, a dye, a nucleic acid control, a reducing agent, dimethyl sulfoxide (DMSO), ethylenediaminetetraacetic acid (EDTA), ethylene glycol-bis(2-aminoethylether)-N,N,N′,N′-tetraacetic acid (EGTA), microspheres capable of binding a nucleic acid or a combination thereof.

41. The assembly of any of embodiments 29-40, wherein the matrix material comprises at least one of a water soluble polymer, a carbohydrate and a combination thereof.

42. The assembly of any of embodiments 29-41, wherein the tablet includes about 1 percent to about 95 percent by tablet weight of the reagent.

43. A method comprising selecting a active component, wherein the active component requires a reconstitution buffer prior to use in a chemical reaction; selecting a substantially solid reconstitution buffer; and forming a tablet comprising the active component and the substantially solid reconstitution buffer, wherein the tablet is sized to fit within at least one chamber of a microfluidic processing device.

44. The method of embodiment 43, wherein the solid reconstitution buffer comprises a nonionic solid surfactant.

45. The method of any of embodiments 43-44, further comprising selecting a matrix material, wherein forming the tablet comprises forming the tablet comprising the active component, the substantially solid reconstitution buffer, and the matrix material.

46. The method of any of embodiments 43-45, wherein the matrix material comprises sorbitol.

47. The method of any of embodiments 43-46, wherein the active component comprises enzymes, primers, and probes.

48. The method of any of embodiments 43-47, further comprising reconstituting the tablet in an aqueous solution.

49. The method of embodiment 48, wherein the aqueous solution comprises water.

50. The method of any of embodiments 43-49, further comprising lyophilizing the reconstitution buffer and active component together prior to forming the tablet.

51. The method of any of embodiments 43-50, further comprising dry mixing the reconstitution buffer and the active component prior to forming the tablet.

52. The method of any of embodiments 43-51, wherein forming the tablet comprises compressing the active component and the reconstitution buffer to define the tablet.

53. An assembly comprising a microfluidic processing device comprising an input chamber and a process chamber fluidically coupled to the input chamber; and a tablet comprising an active component and a substantially solid reconstitution buffer, wherein the tablet is configured to fit within the process chamber of the microfluidic processing device.

54. A method comprising introducing an analyte into a microfluidic sample processing device; and at least partially dissolving a tablet in a chamber of the microfluidic device, wherein the tablet comprises an active component and a substantially solid reconstitution buffer, and is configured to fit within the chamber of the microfluidic processing device.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic top view of an exemplary processing assembly according to the present invention.

FIG. 2 is a flow chart illustrating an exemplary technique for making a tablet according to the present invention.

FIG. 3 is a schematic top view of another exemplary processing assembly according to the present invention.

FIGS. 4A and 4B are magnified views of a process chamber of the exemplary processing assembly of FIG. 3.

FIG. 5 is a partial cross-sectional view of FIG. 4A.

FIG. 6 is a flow chart illustrating an exemplary technique for using a tablet according to the present invention.

FIGS. 7A and 7B are schematic views of a process chamber of an exemplary process assembly including two tablets.

FIG. 8 is a partial cross-sectional view of FIG. 7A.

FIG. 9 is a schematic illustration of exemplary tablet according to the present invention including two layers.

FIG. 10 is a schematic illustration of a processing device including a plurality of sequentially arranged process chambers.

FIG. 11 is a schematic top view of an exemplary processing device according to the present invention.

### DETAILED DESCRIPTION

As described herein, a processing device includes a tablet comprising a reagent. A “tablet” refers to any substantially compressed dosage form of a reagent, and, as described in further detail below, the tablet may include other components in addition to the reagent. The dosage may include a sufficient amount of the reagent for one reaction or for multiple reactions. The processing device may be any suitable substantially self-contained processing device that may receive a sample or other supply of fluid and conduct a particular procedure, such as the preparation of a biological sample for, for example, DNA sequencing, and/or detection, with the aid of one or more chemicals. As other examples, the processing device may be useful for conducting chemical, biological or biochemical reactions. Examples of such reactions include detection via thermal processing techniques, such as, but not limited to, enzyme kinetic studies, homogeneous ligand binding assays, and more complex biochemical or other processes that require precise thermal control and/or rapid thermal variations.

Examples of sample preparation techniques include nucleic acid manipulation techniques, such as, but not limited to, polymerase chain reaction (PCR); target polynucleotide amplification methods such as self-sustained sequence replication (3SR) and strand-displacement amplification (SDA); methods based on amplification of a signal attached to the target polynucleotide, such as “branched chain” DNA amplification; methods based on amplification of probe DNA, such as ligase chain reaction (LCR) and QB replicase amplification (QBR); transcription-based methods, such as ligation activated transcription (LAT), nucleic acid sequence-based amplification (NASBA), amplification under the trade name INVADER, and transcriptionally mediated amplification (TMA); and various other amplification methods, such as repair chain reaction (RCR) and cycling probe reaction (CPR).

A substantially self-contained processing device may include a biological reagent in a controlled amount, thereby eliminating the need for an end-user or another user to measure and introduce the biological reagent into the processing device. For example, one type of microfluidic analytical device is manufactured for dedicated assays and must be pre-packaged with specific reagent chemistries. Such chemistries include biological reagents, buffers and surfactants.

Biological reagents may be expensive and subject to degradation during preparation, storage, and/or use of a processing device. The physical form in which the reagents are introduced into a processing device may have a considerable impact on the manufacturing throughput and shelf life of a processing device containing a reagent.

Conventional techniques for introducing a reagent into processing devices include preparing a reagent in aqueous form and introducing the reagent into the processing device in the form of an aqueous reagent solution. For example, one technique involves the fluidized bed coating of reagents onto inert water soluble spheres. The aqueous reagent solution is dried after being introduced in the sample processing device, thereby resulting in a dried form of the reagent, such as a powder, in the sample processing device. In general, this process of introducing an aqueous reagent solution into a processing device is time-consuming because it requires time for the aqueous reagent solution to dry once the aqueous solution is introduced into the sample processing device.

In addition, a large amount of aqueous reagent solution may be required to deliver a relatively small amount of dried form reagent because the solubility of a reagent in the carrier liquid may dictate the amount of reagent deliverable per volume of liquid solution. In some cases, depending on the type of processing device, a chamber into which the liquid reagent is introduced may be relatively small in volume, which limits the total volume of aqueous reagent solution that can be introduced into the chamber. As a result, the amount of reagent that a processing device may include may be quite limiting when a reagent is placed in the processing device in liquid form.

Moreover, an aqueous reagent solution may be difficult to control. For example, upon introduction of an aqueous reagent solution into a chamber of a processing device, the reagent solution may flow outside the boundaries of a chamber and into a liquid transfer conduit connected to the chamber or an adjacent chamber, which may contaminate the conduit or chamber and negatively influence the desired process. It may also be difficult to introduce a liquid reagent solution into a chamber of a processing device with substantial accuracy and precision, which may be desirable, particularly in the case of a processing device that include relatively small chambers and/or a plurality of relatively small chambers within close proximity to each other.

The problem of controlling the liquid reagent solution may be compounded when more than one type of reagent is introduced into the processing device. In some cases in which a processing device includes more than one type of reagent, it may be desirable to minimize contact between the reagents, e.g., to limit cross-contamination. For example, a single assay may require multiple reagents that are spatially separated for sequential use. Because of the potentially large volume of reagents and flow properties of liquids, introduction of reagents in the form of aqueous solutions may prevent the precision spotting of multiple reagents within a single chamber of a sample processing device. In addition, aqueous forms of reagents, in general, can be particularly difficult to handle due to their limited stability in solution at room temperature.

Some current techniques for introducing biological reagents into a processing device rely on a dried form of the reagent. For example, biological reagents may be produced via dry-blending, spray drying, freeze-drying, fluidized bed drying, and cryogenic freezing. It is desirable for reagents to be introduced into a processing device in analytically precise amounts. Several dosage forms have been proposed to achieve this including freeze-dried spheres, and aqueous paste extrusion and pelletization. However, each of these methods suffers from drawbacks such as cost, slow aqueous reconstitution in the device, a lack of mechanical stability and stability during storage of the processing device. In addition, these non-compressed dosage forms or reagent forms that are not dimensionally stable may be relatively difficult to handle and store, particularly compared to a reagent tablet that is mechanically and dimensionally stable. For example, freeze-dried spheres with a relatively high concentration of reagents may be difficult to prepare and handle in terms of dimensional and mechanical stability. Additionally, freeze-dried spheres may be relatively large in comparison to the requirements for use in a processing device because the components have not been compressed. In some cases, size requirements dictated by a processing device, such as a microfluidic device, may limit the size of a freeze-sphere that can be introduced into a microfluidic device, thereby limiting the amount of reagent that may be introduced.

The present invention addresses at least some of the previous drawbacks of the prior dried reagents forms. In particular, in accordance with the present disclosure, a processing device includes a biological reagent in a tablet form. A tablet including a reagent in a substantially compact configuration is more mechanically stable than, e.g., a reagent in a liquid or powder form, or a lyophilized pellet of a reagent, or even a support film including a reagent layer. A mechanically stable reagent tablet may permit easier handling, e.g., manually or by a robotic arm or another computer-controlled apparatus, during a process in which the tablet is assembled with the processing device. As a result of the mechanical stability of the tablet, the tablet is substantially dimensionally stable. In some embodiments, the tablet substantially maintains its shape and dimensions within about 5%, such as about 1%, during handling and introduction of the tablet into the processing device. In contrast, a reagent in a liquid form or a powder form that is not compact or otherwise defines a common structure that can be handled by a robotic arm, may not be considered substantially dimensionally stable. The mechanical and dimensional instability of the liquid or powder reagent may be difficult to integrate into an automated manufacturing process, due to, for example, dry time of the liquid reagent, an increased potential contamination between different chambers of the processing device, and so forth.

It has been found that compressing a biological reagent, such as lysostaphin, into a tablet form does not substantially destroy the reagent or the usefulness of the reagent in a particular reaction. As demonstrated by the Examples given below, it has been found that the compressing a reagent, such as lysostaphin, and matrix material at relatively high compression pressures, e.g., pressures of about 15 megapascals (MPa) to about 200 MPa, does not affect the ability of the reagent to react with an analyte.

The amount of reagent in a tablet may be varied. For example, the size of the tablet, the amount of matrix material used in the tablet, and the type of reagent material may be varied to adjust the amount of reagent within a tablet. In general, the amount of reagent present in tablet is at least a suitable amount for use in a processing device. In some embodiments, a tablet may include about 0.1 percent (%) by tablet weight to about 99.9% by tablet weight of reagent in the tablet, such as about 1% to about 99% by tablet weight. For example, in certain embodiments, the reagent in a tablet may range from about 1% to about 95% by tablet weight, such as about 50% to about 95%. In some embodiments, the reagent in a tablet may range from about 50% to about 90% by tablet weight, such as about 75% to about 80%. Certain embodiments may also allow for microtablets with relatively very high percentages of active components (e.g., reagents or other components that may react with analyte), which may also be referred to as “reactive” components, even with components that are poorly compressible in macroscopic form. In addition, in some embodiments, a tablet may include more than one type of reagent.

In some embodiments, the solubility of a tablet may be controlled by some or all of the tablet components. In some embodiments, a tablet may include a matrix material having a solubility of about 0 grams per approximately 100 grams of water to about 1000 grams per approximately 100 grams of water. In other embodiments, a tablet may include a matrix material having a solubility of about 0 grams per approximately 100 grams of water to about 400 grams per approximately 100 grams of water.

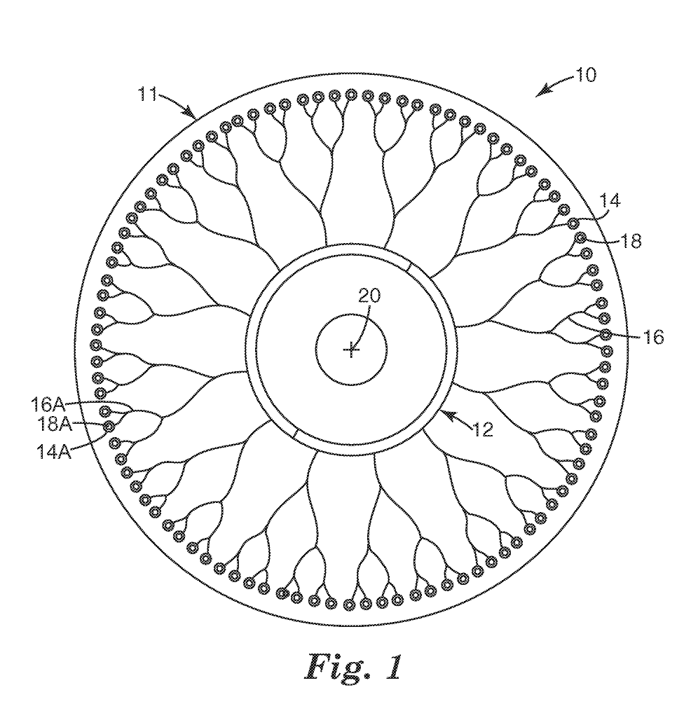

FIG. 1 is a schematic top view of an exemplary processing assembly 10 that includes a processing device 11 including loading chamber 12, a plurality of process chambers 14, and a plurality of conduits 16 coupling loading chamber 12 with at least one process chamber 14. Process chambers 14 each define a volume for containing a fluid or a conduit through which a fluid may pass through (e.g., capillaries, passageways, conduits, grooves). A tablet 18 is disposed within each of process chambers 14. In the embodiment shown in FIG. 1, conduits 16 are each a microfluidic conduit. Thus, processing device 11 may also be referred to as a “microfluidic processing device.”

Processing assembly 10 is useful for processing an analyte, which may be in the form of a fluid (e.g., a solution, etc.) or a solid or semi-solid material carried in a fluid. For example, processing device 10 may include a chemical component (e.g., tablet 18) that is useful for preparing an analyte for detection of a particular bacteria or other target microorganism of interest within the analyte. The analyte may be from a living (e.g., a human patient) or nonliving source (e.g., a food preparation surface). The analyte may be entrained in the fluid, in solution within the fluid, and so forth. Thus, reference to an “analyte” or “sample” refers to any fluid in which the analyte is or may be located, regardless of whether the analyte is, itself, a fluid or is contained within a carrier fluid (in solution, suspension, etc.). Furthermore, in some instances, analyte may be used to refer to fluids in which a target analyte (i.e., the analyte sought to be processed) is not present. For example, wash fluids (e.g., saline, etc.) may also be referred to as an analyte.

A user may introduce an analyte into loading chamber 12, which may then be introduced into at least one of process chambers 14 via the respective conduit 16. Any suitable technique may be employed to move the analyte from loading chamber 12 to the respective process chamber 14, such as via centrifugal forces generated by rotating processing device 10 about a center axis 20, gravitational forces (actual or induced), thermal transfer techniques, as described in commonly-assigned U.S. Patent Application Ser. No. 60/871,611 (Bedingham et al.) filed on Dec. 22, 2006, which is incorporated herein by reference in its entirety, or other suitable techniques. Although movement of fluids within processing device 10 is primarily described with reference to centrifugal forces generated by rotation of device 10, in other embodiments, any one or combination of techniques may be used to move fluid within processing device 11, e.g., a combination of rotational and gravitational forces.

After moving into one or more process chambers 14, the analyte may be processed to obtain a desired reaction, such as, but not limited to a polymerase chain reaction (PCR), ligase chain reaction (LCR), sustaining sequence replication, enzyme kinetic studies, homogeneous ligand binding assays, and other chemical, biochemical, or other reactions. A “chamber” as used herein should not be construed as limiting the chamber to one in which a process (e.g., PCR, Sanger sequencing, etc.) is performed. Rather, a chamber may include, e.g., a volume in which materials are loaded for subsequent delivery to another chamber as the processing device if rotated, a chamber in which the product of a process is collected, a chamber in which materials are filtered, and so forth.

In the embodiment shown in FIG. 1, upon introduction into at least one of the chambers 14, the analyte reacts with a reagent within tablet 18. Process chamber 14A, conduit 16A, and tablet 18A are primarily referred to throughout the description of FIG. 1, however, the description of process chamber 14A, conduit 16A, and tablet 18A are also applicable to each of the plurality of process chambers 14 and respective conduits 16 and tablets 18. Tablets 18 may or may not include the same composition.

In the embodiment shown in FIG. 1, tablet 18A includes at least one type of reagent and a matrix material that may or may not be soluble. Fluid from the analyte or a fluid otherwise introduced into process chamber 14A may be used to at least partially dissolve tablet 18A and release the reagent therein. In order to increase the speed of the dissolution of tablet 18A, processing device 11 may be manipulated to encourage fluid flow around tablet 18A. For example, processing device 11 may be rotated about center axis 20 in a particular pattern (e.g., accelerating or decelerating in a particular pattern). As another example, vacuum forces may be introduced into chambers 14 via sample loading chamber 12 or another source, and the release and application of the vacuum force may encourage the movement of fluid within process chamber 14A.

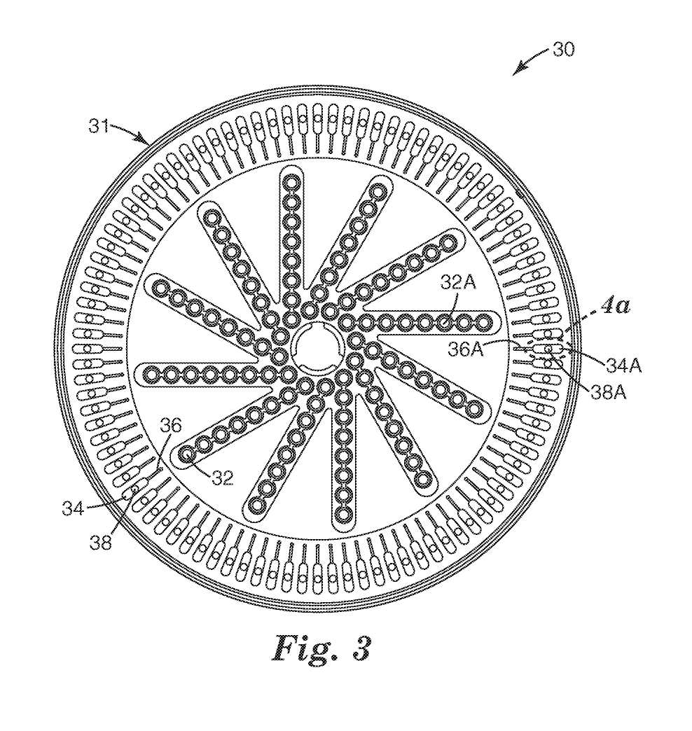

While processing device 10 is shown in FIG. 1 to have a circular disc shape, in other embodiments, processing device 10 may define any other suitable shape. In some embodiments, a shape of processing device 10 is selected to aid rotation of device 10. In addition, processing device 10 may include any suitable number of process chambers 14 and supply chambers 12. For example, while 96 process chambers 14 are shown in FIG. 1, in other embodiments, a processing device may include as few as one process chamber or more than 96 process chambers. Furthermore, in other embodiments, a process chamber may include multiple supply chambers, e.g., as shown and described below with respect to FIG. 3

In some embodiments, processing device 10 may be a thermal transfer structure. The thermal transfer processing device 10 may be useful for reactions that require relatively precise thermal control (e.g., an isothermal process sensitive to temperature variations) and/or rapid thermal variations. It may be preferred that at least one of the sides of the processing device 11 present a surface that is complementary to a base plate or thermal structure apparatus as described in, e.g., U.S. Pat. No. 6,734,401 titled ENHANCED SAMPLE PROCESSING DEVICES SYSTEMS AND METHODS (Bedingham et al.); U.S. Patent Application Publication No. 2007/0009391, titled COMPLIANT MICROFLUIDIC SAMPLE PROCESSING DISKS, filed on Jul. 5, 2005; and U.S. Patent Application Publication No. 2007/0010007, titled SAMPLE PROCESSING DEVICE COMPRESSION SYSTEMS AND METHODS, filed on Jul. 5, 2005. In some embodiments, it may be preferred that at least one of the major sides of the processing devices of the present invention present a flat surface.

A tablet according the present invention, such as tablet 18A, may include at least one reagent which can be used in at least one step of an analytical procedure, including sample preparation and detection steps. Non-limiting examples include polynucleotide or nucleic acid manipulation techniques or protein processing. In some embodiments, a tablet may include reagents generally used for polymerase chain reaction. For certain embodiments, including any one of the above embodiments, a tablet includes at least one reagent that can be used in at least one of a step of sample preparation, a step of nucleic acid amplification, a step of detection in a process for detecting or assaying a nucleic acid and a step of detection in a process for detecting or assaying a amino acid. Sample preparation may include, for example, capturing a biological material containing a nucleic acid, washing a biological material containing a nucleic acid, lysing a biological material containing a nucleic acid, for example, cells or viruses, digesting cellular debris, isolating, capturing, or separating at least one polynucleotide or nucleic acid from a biological sample, and/or eluting a nucleic acid. Nucleic acid amplification may include, for example, producing a complementary polynucleotide of a polynucleotide or a portion of a nucleic acid in sufficient numbers for detection. Detection includes, for example, making an observation, such as detecting a fluorescence, which indicates the presence and/or amount of a polynucleotide or nucleic acid.

In some embodiments, tablet 18A includes at least one reagent selected from the group consisting of a lysis reagent, a protein-digesting reagent, a nucleic acid amplifying enzyme, an oligonucleotide, a probe, nucleotide triphosphates, a buffer, a salt, a surfactant, a dye, a nucleic acid control, a reducing agent, dimethyl sulfoxide (DMSO), glycerol, ethylenediaminetetraacetic acid (EDTA), ethylene glycol-bis(2-aminoethylether)-N,N,N′,N′-tetraacetic acid (EGTA), microspheres capable of binding a nucleic acid, and a combination thereof. In addition, in some embodiments, a group of reagents from which the at least one reagent is selected further includes any one of, any combination of, or all of RNase, DNase, an RNase inhibitor, a DNase inhibitor, Bovine Serum Albumin, spermidine, and a preservative.

As previously described, processing assembly 10 including device 11 and tablets 18 may be useful for sample preparation, such as lysis. Lysis can be accomplished enzymatically, chemically, and/or mechanically. Enzymes used for lysis include, for example, lysostaphin, lysozyme, mutanolysin or others. Chemical lysis can be carried out using a surfactant, alkali, heat, or other means. When alkali is used for lysis, a neutralization reagent may be used to neutralize the solution or mixture after lysis. Mechanical lysis can be accomplished by mixing or shearing using solid particles or microparticles such as beads or microbeads. The lysis reagent can include a surfactant or detergent such as sodium dodecylsulfate, lithium dodecylsulfate, or N-methyl-N-(1-oxododecyl)glycine, sodium salt, or the like, buffered as needed; a chaotrope such as guanidium hydrochloride, guanidium thiacyanate, sodium iodide, or the like; a lysis enzyme such as lysozyme, lysostaphin, mutanolysin, proteinases, pronases, cellulases, or any of the other commercially available lysis enzymes; an alkaline lysis reagent; a neutralization reagent, solid particles such as beads, or a combination thereof.

The protein-digesting reagent can facilitate digestion of proteins present in the sample material, including a lysis enzyme if present. In addition, the protein-digesting reagent, for example, proteinase K, can act as a lysis reagent in the presence of a surfactant.

“Nucleic acid amplifying enzyme” refers to an enzyme which can catalyze the production of a polynucleotide or a nucleic acid from an existing DNA or RNA template. In some embodiments, tablet 18A includes a nucleic acid amplifying enzyme that can be used in a process for amplifying a nucleic acid or a portion of a nucleic acid. For example, in some embodiments, the nucleic acid amplifying enzyme is selected from the group consisting of a DNA polymerase and a reverse transcriptase. In other embodiments, the DNA polymerase is selected from the group consisting of Taq DNA polymerase, Tfl DNA polymerase, Tth DNA polymerase, Tli DNA polymerase, and Pfu DNA polymerase. For certain of these embodiments, the reverse transcriptase is selected from the group consisting of AMV reverse transcriptase, M-MLV reverse transcriptase, and M-MLV reverse transcriptase, RNase H minus. Retroviral reverse transcriptase, such as M-MLV and AMV posses an RNA-directed DNA polymerase activity, a DNA directed polymerase activity, as well as an RNase H activity. For certain embodiments, the nucleic acid amplifying enzyme is a DNA polymerase or an RNA polymerase. For certain embodiments, the nucleic acid amplifying enzyme is Taq DNA polymerase. For certain embodiments, the nucleic acid amplifying enzyme is T7 RNA polymerase.

In some embodiments in which tablet 18A includes an “oligonucleotide,” the oligonucleotide may be a primer, a terminating oligonucleotide, an extender oligonucleotide, or a promoter oligonucleotide. For certain embodiments, the oligonucleotide is a primer. Such oligonucleotides may be comprised of 15 to 30 nucleotide units, which determine the region (targeted sequence) of a nucleic acid to be amplified. Under appropriate conditions, the bases in the primer bind to complementary bases in the region of interest, and then the nucleic acid amplifying enzyme extends the primer as determined by the targeted sequence. A large number of primers are known and commercially available, and others can be designed and made using known methods.

In some embodiments, tablet 18A may include a probe that allows detection of amplification products (amplicons) by fluorescing, and thereby generating a detectable signal, the intensity of which is dependent upon the number of fluorescing probe molecules. Probe molecules can be comprised of an oligonucleotide and a fluorescing group coupled with a quenching group. Probes can fluoresce when separation or decoupling of the quenching group and the fluorescing group occurs upon binding to an amplicon or upon nucleic acid amplifying enzyme cleavage of the probe bound to the amplicon. Alternatively, a probe bound to the amplicon can fluoresce upon exposure to light of an appropriate wavelength. For certain embodiments, including any one of the above embodiments, the probe is selected from the group consisting of TAQMAN probes (available from Applied Biosystems, Foster City, Calif.), molecular beacons, SCORPIONS probes (available from Eurogentec Ltd., Hampshire, United Kingdom), SYBR GREEN (available from Invitrogen, Carlsbad, Calif.), FRET hybridization probes (available from Roche Applied Sciences, Indianapolis, Ind.), Quantitect probes (available from Qiagen, Valencia, Calif.), and molecular torches.

The nucleotide triphosphates (NTPs), including ribonucleotide triphosphates and deoxyribonucleotides triphosphates as required, are used by the nucleic acid amplifying enzyme in the production of a polynucleotide or a nucleic acid from an existing DNA or RNA template. For example, when amplifying a DNA, a dNTP (deoxyribonucleotide triphosphate) set is used, which typically includes dATP (2′-deoxyadenosine 5′-triphosphate), dCTP (2′-deoxycytodine 5′-triphosphate), dGTP (2′-deoxyguanosine 5′-triphosphate), and dTTP (2′-deoxythimidine 5′-triphosphate).

In some embodiments, tablet 18A may include a buffer. Buffers are used to regulate the pH of the reaction media. A wide variety of buffers are known and commercially available. For example, morpholine buffers, such as 2-(N-morpholino)ethanesulfonic acid (MES), can be suitable for providing an effective pH range of about 5.0 to about 6.5, imidazole buffers can be suitable for providing an effective pH range of about 6.2 to about 7.8, and tris(hydroxymethyl)aminomethane (TRIS) buffers and certain piperazine buffers such as N-(2-hydroxyethyl)piperazine-N′-(2-ethanesulfonic acid) (HEPES) can be suitable for providing an effective pH range of about 7.0 to about 9.0. The buffer can affect the activity and fidelity of nucleic acid amplifying enzymes, such as polymerases. For certain embodiments, the buffer is selected from at least one buffer which can regulate the pH in the range of about 7.5 to about 8.5. For certain of these embodiments, the buffer is a TRIS-based buffer. For certain of these embodiments, the buffer is selected from the group consisting of at least one of TRIS-EDTA, TRIS buffered saline, TRIS acetate-EDTA, and TRIS borate-EDTA. Other materials can be included with these buffers, such as surfactants and detergents, for example, CHAPS or a surfactant described below. For certain embodiments, the buffers are free of RNase and DNase.

Salts can affect the activity of nucleic acid amplifying enzymes. Accordingly, in some embodiments, tablet 18A may include a salt. For example, free magnesium ions are necessary for certain polymerases, such as Taq DNA polymerase, to be active. In another example, in the presence of manganese ions, Tfl DNA polymerase and Tth DNA polymerase can catalyze the polymerization of nucleotides into DNA, using RNA as a template. In a further example, the presence of certain salts, such as potassium chloride, can increase the activity of certain polymerases such as Taq DNA polymerase. For certain embodiments, including any one of the above embodiments, the salt is selected from the group consisting of at least one of magnesium, manganese, zinc, sodium, and potassium salts. For certain of these embodiments, the salt is at least one of magnesium chloride, manganese chloride, zinc sulfate, zinc acetate, sodium chloride, and potassium chloride. For certain of these embodiments, the salt is magnesium chloride.

In some embodiments, tablet 18A may include a surfactant. A surfactant may be useful for lysing or de-clumping cells, improving mixing, enhancing fluid flow, for example, in a device, such as a microfluidic device. The surfactant can be non-ionic, such as a poly(ethylene oxide)-polypropylene oxide) copolymer available, for example, under the trade name PLURONIC, polyethylene glycol (PEG), polyoxyethylenesorbitan monolaurate available under the trade name TWEEN 20, 4-(1,1,3,3-tetramethylbutyl)phenyl-polyethylene glycol available under the trade name Triton X-100; anionic, such as lithium lauryl sulfate, N-lauroylsarcosine sodium salt, and sodium dodecyl sulfate; cationic, such as alkyl pyridinium and quaternary ammonium salts; zwitterionic, such as N-(C₁₀-C₁₆alkyl)-N,N-dimethylglycine betaine (in the betaine family of surfactants); and/or a fluoro surfactant such as FLUORAD-FS 300 (3M, St. Paul, Minn.) and ZONYL (Dupont de Nemours Co., Wilmington, Del.).

In some embodiments, a dye can be included in tablet 18A to impart a color or a fluorescence to the tablet or to a fluid which contacts the tablet. The color or fluorescence can provide visual evidence or a detectable light absorption or light emission evidencing that tablet 18A has been dissolved, dispersed, or suspended in the fluid which contacts the tablet. For certain embodiments, the dye is selected from the group consisting of fluorescent dyes, such as fluorescein, cyanine (which includes Cy3 and Cy5), Texas Red, ROX, FAM, JOE, SYBR Green, OliGreen, and HEX. In addition to these fluorescent dyes, ultraviolet/visible dyes, such as dichlorophenol, indophenol, saffranin, crystal violet, and commercially-available food coloring can also be used.

In some embodiments, tablet 18A may include a nucleic acid control, which is a known amount of a nucleic acid or nucleic acid containing material dried-down with either the sample preparation or the amplification or detection reagents. This internal control can be used to monitor reagent integrity as well as inhibition from the sample material or specimen. For example, linearized plasmid DNA control may be used as a nucleic acid internal control.

In some embodiments, tablet 18A may include a reducing agent, which is a material capable of reducing disulfide bonds, for example in proteins which can be present in a sample material or specimen, and thereby reduce the viscosity and improve the flow and mixing characteristics of the sample material. For certain embodiments, the reducing agent preferably contains at least one thiol group. Examples of reducing agent include N-acetyl-L-cysteine, dithiothreitol, 2-mercaptoethanol, and 2-mercaptoethylamine.

In some embodiments, tablet 18A may include other materials, such as, but not limited to, dimethyl sulfoxide (DMSO), which can be used to inhibit the formation of secondary structures in the DNA template; glycerol, which can improve the amplification process, can be used as a preservative, and can stabilize enzymes such as polymerases; ethylenediaminetetraacectic acid (EDTA) and ethylene glycol-bis(2-aminoethylether)-N,N,N′N′-tetraacetic acid (EGTA), which can be used as metal ion chelators and also to inactivate metal-binding enzymes (RNAses) that may damage the reaction; a passive reference dye, such as ROX; and reagents to amplify nucleic acids in a PCR reaction, including: buffers such as HEPES or Tris-based; salts such as magnesium and potassium-based; and carrier proteins, such as Bovine Serum Albumin (BSA).

In some embodiments, tablet 18A may include RNase or DNase, which may be useful for breaking down undesired RNA or DNA which is present in a sample material. For example, when DNA is being targeted, RNA which may be present can be rendered non-interfering with RNase; and likewise, when RNA is being targeted, DNA which may be present can be rendered non-interfering with DNase. Alternatively, when RNase and/or DNase may be present, but are undesired because of their ability to break down a targeted RNA or DNA, an RNase inhibitor or a DNase inhibitor or both may be used to prevent such break down.

In some embodiments, tablet 18A may include Bovine Serum Albumin, which can be used to stabilize the nucleic acid amplifying enzyme during nucleic acid amplification. In addition, if processing device 10 is used for DNA amplification, in some embodiments, tablets 18 may include certain compounds to stimulate the amplifying enzyme. For example, spermidine may be used to stimulate RNA polymerase.

In some embodiments, tablet 18A according to the present invention may include a preservative to inhibit or prevent inadvertent microbial growth in the tablet. For example, a synthetic preservative such as methyl paraben, propyl paraben, sodium azide, or the like may be used for this purpose.

In some embodiments, tablet 18A may include microspheres. The term “microspheres” refers to microspheres, microparticles, microbeads, resin particles, and the like. Microspheres capable of binding a nucleic acid can be useful in a sample preparation step where, for example, at least one polynucleotide or nucleic acid is isolated or separated from a biological sample. Examples of microspheres capable of binding a polynucleotide or nucleic acid include resin and silica particles with metal ions immobilized on the surface of the resin or silica particles. Resin particles can be latex beads, polystyrene beads, and the like. The resin or silica particles can be magnetic or non-magnetic. The particles can be colloidal in size, for example about 100 nanometers (nm), to about 10 micrometers (μm). Such immobilized metal resin particles can be made as described in U.S. Pat. No. 7,112,552 at Examples 1 and 2; U.S. Patent Application Publication No. 2004/0152076 at paragraph 0152, and in U.S. Provisional Patent Application No. 60/913,812, entitled COMPOSITIONS, METHODS, AND DEVICES FOR ISOLATING BIOLOGICAL MATERIALS, filed on Apr. 25, 2007 (Xia et al.). Microspheres can also be used for resuspension and mixing of sample preparation, amplification, or detection reagents. For example, glass or magnetic beads without or with binding capability can be used for this purpose.

For certain embodiments, including any one of the above embodiments, tablet 18A according to the present invention may include at least one reagent selected from the group consisting of a nucleic acid amplifying enzyme, a primer, a probe, and microspheres capable of binding a nucleic acid. For certain embodiments, including any one of the above embodiments, tablet 18A may include at least one reagent selected from the group consisting of a nucleic acid amplifying enzyme, a primer, and a probe. For certain of these embodiments, the tablet includes a nucleic acid amplifying enzyme. In these embodiments, nucleic acid amplifying enzyme, primer, and probes can include any one of the embodiments described above for each of these reagents.

For certain embodiments, including any one of the above embodiments, tablet 18A according the present invention may further include a matrix material. In some embodiments, the matrix material is selected from the group consisting of a water soluble polymer, a carbohydrate and a combination thereof. As used herein, “water soluble” means that material, for example, the water soluble polymer, carbohydrate, or a combination thereof, can be dissolved, dispersed, or suspended in water at a temperature that is at least room temperature. For certain embodiments, the temperature is at least about 50° C. For certain embodiments, the temperature is not more than about 100° C., such as not more than about 97° C. or not more than about 75° C. The matrix material can hold or contain at least one reagent. The matrix material can also increase cohesion of the tablet components and stabilize the dimensions within the tablet. In some embodiments the matrix material is an active component of the tablet. For example, a matrix material, such as sorbitol, may help protect a particular enzyme within an aqueous solution. In other embodiments the matrix material is an inert component within the tablet. In certain embodiments, the matrix material is an excipient.

For certain of these embodiments, the matrix material is a water soluble polymer. For example, the water soluble polymer may be selected from the group consisting of poly(ethylene glycol), poly(vinyl alcohol), partially hydrolyzed poly(vinyl alcohol), polyvinylpyrrolidone, poly(1-vinylpyrrolidone-co-2-dimethylaminoethylmethacrylate), poly(1-vinylpyrrolidone-co-vinyl acetate), and a combination thereof. As other examples, the water soluble polymer is selected from the group consisting of poly(vinyl alcohol), poly(vinyl alcohol acetate), polyvinylpyrrolidone, and a combination thereof. As yet other examples, the water soluble polymer is poly(vinyl alcohol) that is at least 80% hydrolyzed, such as a poly(vinyl alcohol) that is at least 90% hydrolyzed and has a weight average molecular weight of about 30,000 to about 70,000.

In other embodiments, tablet 18A includes a matrix material that includes a carbohydrate. For example, the carbohydrate may be selected from the group consisting of sucrose, trehalose, mannitol, sorbitol, raffinose, stachyose, melezitose, dextrose, maltose, dextran, cellobiose, pectin, hydroxyethylcellulose, hydroxypropylcellulose, hydroxypropylmethylcellulose, guar gum, locust gum, gum arabic, xanthan gum, ficoll, a poly(ethylene oxide)-poly(propylene oxide) copolymer with a hydrophilic/lipophilic balance of greater than 7, preferably greater than 9, more preferably about 12, a cyclodextrin, α-cyclodextrin, starch, pullulan, alginates, gelatins, and carrageenans. As other examples, the carbohydrate may at least one of sucrose, dextran, trehalose, pullulan, α-cyclodextrin, mannitol, sorbitol, and a combination thereof.

For some embodiments in which the matrix material is a carbohydrate, the carbohydrate is a sugar. A matrix material sugar may be either reducing or non-reducing. In general, a reducing sugar is a sugar may be any sugar that, in basic solution, forms some aldehyde or ketone, thereby allowing the sugar to act as a reducing agent. A reducing sugar may also refer to sugars that react with Tollens', Benedict's or Fehling's reagents. Reducing sugars include, for example, glucose, glyceraldehyde, lactose, arabinose and maltose. In general, nonreducing sugars include sugars that do not substantially react with Tollens', Benedict's or Fehling's reagents, such as, for example, trehalose.

For some of these embodiments, the matrix material includes a combination of a water soluble polymer and a carbohydrate. In these embodiments, the water soluble polymer and the carbohydrate can be independently selected from any one of the above embodiments.

In some embodiments, tablet 18A may also include a lubricant material, such as, but not limited to, leucine, valine, polyethylene Glycol (PEG), magnesium stearate, stearic acid, sodium stearate, sodium stearyl fumarate, sodium lauryl sulfate, micronized pluronics (e.g., lubricants available under the trade names Lutrol 68, Lutrol 127 from BASF Aktiengesellschaft of Ludwigshafen, Germany). Certain reagents, such as lysostaphin, may also provide lubricant properties, thereby minimizing or even eliminating the need to add an addition lubricant to tablet 18A. A lubricant material may be useful during a tablet formation process, such as by preventing adhesion between tablet 18A and another surface in which the tablet may contact. For example, a lubricant material may minimize or even prevent adhesion between a tablet surface and a compression device used to form tablet 18A, such as, for example, a tablet press. Tablet 18A for use with processing device 11 according to the present invention can further include additional components, such as fillers and plasticizers. If included, additional optional components are used in minimal amounts and, preferably do not interfere with the activity or function of any of the reagents.

In some embodiments, tablet 18A for use with processing device 11 according to the present invention may include one or more disintegrants. Disintegrants may be used to aid in the dissolution process. For example, a disintegrant may act to increase the dissolution rate of a tablet in a fluid of a processing device. In some embodiments, a disintegrant may be insoluble or have relatively low soluble in a fluid of a processing device. In such embodiments, distintegrant particles not dissolved in a fluid of a processing device may have to be spun down within a processing device so that they do not interfere with one or more reactions within the device. Examples of disintegrants include starch, sodium starch glycolate (explotab and primogel), cross-linked polyvinyl pyrrolidone (cross povidone), alginates, purified cellulose, methylcellulose, crosslinked sodium carboxy methylcellulose (Ac-Di-Sol), carboxy methyl cellulose, cross carmellose sodium, microcrystalline cellulose (Avicel pH-101, pH-102, pH-105), Ambrelite IPR 88 (Ion Exchange Resins), gums such as agar, locust bean, karaya, Pectin and tragacanth, guar gums, gum karaya, Chitin and Chitosan, Smecta, Gellan gum, Isapghula Husk, Polacrillin Potassium (Tulsion³³⁹), and agar. Exemplary disintegrants may also include gas-evolving disintegrants, which may involve the inclusion of citric acid and tartaric acid along with sodium bicarbonate, sodium carbonate, potassium bicarbonate or calcium carbonate. Typically, gas-evolving disintegrants may react in contact with water to liberate carbon dioxide that disrupts the tablet. Some distintegrants may be classified as “superdisintegrants”. Generally, a superdisintegrant may increase the dissolution rate of a tablet even more than regular disintegrants. Examples of superdisintegrants include crosscarmelose, crosspovidone, sodium starch glycolate which represent example of a crosslinked cellulose, crosslinked polymer and a crosslinked starch.

In some embodiments, a manufacturer may provide an active component of a reagent in a powder form, along with a substance, such as a reconstitution buffer, with the understanding that the active component will not serve its intended purpose in a reaction until reconstituted via the substance. To facilitate relatively long-term storage and manipulation of the active components, it may be convenient to reduce the active components to solid formulations (e.g., a powder) that can be easily reconstituted with an aqueous reconstitution buffer prior to use. In the case of such active components, the active components may be reconstituted in a reconstitution buffer before being introduced into a processing device and/or before being used in a reaction. In general, reconstitution buffers solubilize active components, such as enzymes, primers, and probes. For example, active components may include reverse transcriptase and RNA polymerase enzymes, active components of amplification reagents, active components of chemiluminescence reagents, and molecular torches. Non-limiting examples of such reagents include enzymes that are stored as lyophilized powders. The enzymes may be reconstituted with a reconstitution buffer that includes other reagent components, such as glycerol and nonionic surfactants.

A reconstitution buffer including a nonionic surfactant may be added to enzyme formulations to help in solubilizing the enzyme and, in some cases, to protect the enzyme from degradation. For example, glycerol may be added to a reconstitution buffer for storage stability of an enzyme in the liquid form. Glycerol may also benefit the performance of the enzyme in terms of affecting the melt temperature of the primers. Other examples of enzymes that may be reconstituted by a liquid reconstitution buffer are described in U.S. Pat. No. 5,556,771 (Shen et al.).

In some embodiments, a tablet may be formed with one or more active components and respective reconstitution buffers. However, certain reconstitution buffers that include nonionic surfactants in a liquid state, such as a nonionic surfactant made available under the trade name Triton X-100 by BASF of Ludwigshafen, Germany, do not lend themselves to being dried down, which is useful for preparing the reconstitution buffer for tabletting. That is, because a liquid reconstitution buffer may be difficult to tablet due to its liquid form, it may be desirable to provide a solid reconstitution buffer as a substitute for the liquid reconstitution buffer. Some reconstitution buffers do not lend themselves to reduction to solid formats as they contain chemical components that are liquid at room temperature.

As a result, forming a tablet with large percentages of Triton X-100 or other liquid reconstitution buffers may not be feasible. Therefore, if a active component is provided a liquid nonionic surfactant, a solid substitute for the nonionic surfactants may be selected prior to tabletting the active components. For example, a nonionic surfactant made available under the trade name Triton X-405 by BASF of Ludwigshafen, Germany or ethylene oxide/propylene oxide block copolymers are useful as additives for tabletting enzymes for TMA assays. Another solid surfactant, Pluronic F127 (polyoxyethylene-polyoxyproplene copolymer) made available by BASF of Ludwigshafen, Germany can also be used as a substitution of glycerol as an enzyme stabilizer in a tablet. The substantially solid nonionic surfactants may be mixed with the active components via any suitable technique. In some embodiments, the substantially solid nonionic surfactant may be either co-lyophilized with the enzyme or blended in as a powder. An additional benefit conferred by some solid surfactants is their ability to function as lubricants for the tabletting process.

In the case of reagents that are intended to be reconstituted by a liquid reconstitution buffer prior to use in a reaction that takes place within a processing device, it may be necessary to substitute a substantially solid reconstitution buffer for the liquid reconstitution buffer prior to tabletting the reagent. It is believed that some substantially solid reconstitution buffers may be sufficient substitutes for certain liquid reconstitution buffers. In some embodiments, a tablet according to the present invention may include a reagent and a substantially solid reconstitution buffer, as well as any other suitable tablet components, such as matrix materials or lubricants. In general, reconstitution of reagents by a substantially solid reconstitution buffer may allow for reconstitution of a reagent without the use of a liquid reconstitution buffer. Substantially solid reconstitution buffers may include suitable solid surfactants, such as, for example, a nonionic surfactant made available under the trade names Triton X-405 or Pluronic F127 by BASF of Ludwigshafen, Germany. In some embodiments, suitable solid surfactants may be nonionic so as to limit the influence on the reaction of the tabletted reagents within processing device 10.

Tablets including substantially solid reconstitution buffers may also include a solid matrix material. For example, a tablet including a substantially solid reconstitution buffer may include sorbitol, a solid polyol sugar. In some embodiments, if a substantially solid reconstitution buffer is substituted for a substantially liquid reconstitution buffer that included glycerol (which is typically a substantially liquid matrix material) as a matrix material, sorbitol may be included in a tablet as a substitute for glycerol in the substantially solid tablet. As other examples, an aqueous reconstitution buffer including glycerol may be substituted with a solid reconstitution buffer that includes other polyhydric alcohols, such as sucrose, glucose, sorbitol, erythritol, pentaerythritol, dextrin, polysaccharides, and maltose. A particularly beneficial cryoprotectant is trehalose.

In some embodiments, surfactants bearing both sugar head groups and oxyethlene chains, such as ANAGRADE® SUCROSE MONODODECANOATE, made available by Anatrace, Inc. of Maumee, Ohio or Big CHAP (N,N′-Bis(3-D-gluconamidopropyl)cholamide) may be used to substitute both glycerol and liquid Triton X-100 in a tabletting format for reagents.

The above described materials, i.e. reagents, matrix materials, lubricant materials, additional materials and disintegrants, as well as any other material included in the composition of a tablet according to the present invention, may be referred to generally as “components of a tablet” or “tablet components.” Components of a tablet according to the present invention, such as those described above, may be either active or inert components. In general, active components include any component that participates in a reaction that takes place within processing device 11. Conversely, an inert component is generally any component that does not interfere or help with the reaction that takes place within processing device 11. Typically, a reagent, such as that listed above, will be an active component of tablet 18A. A matrix material, such as that listed above, may be either an active component or inert component. Components may be selected, at least in part, based on whether a component will be active or inert within a processing device.

Tablet 18A according to the present invention may include varying amounts of active components. In some embodiments, tablet 18A may include about 100% active components by weight, i.e., a tablet with only active components and substantially no inert components. In other embodiments, the amount of active components may be less than about 100% of total tablet weight. For example, the amount of active components in a tablet may range from about 0.1% to 99% by weight of the tablet, such as about 1% to about 95% by total tablet weight. In general, the percent of active components in tablet 18A may be varied depending on the amount of active components desired to be introduced to processing device 11, such as, for example, into process chamber 14 of processing device 11. In some cases, the amount of active components in a tablet may be similar to the ranges provided above with respect to the percentages of reagents within a tablet.

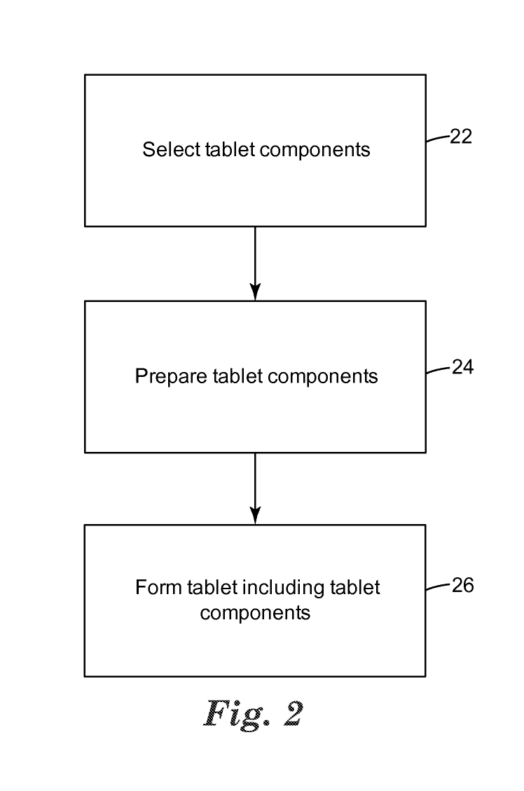

FIG. 2 is a flow chart illustrating an exemplary technique to form tablet 18A according to the present invention. Tablet components desired in tablet form are selected (22). As described above, tablet 18A may include components such as one or more reagents, matrix materials, and lubricant materials. Selection of tablet components (22) may involve one or more considerations. For example, considerations such as the type of reaction to be performed within the particular process chamber 14 of processing device 11, the type of fluid, such as the type of analyte, that will be introduced in processing device 11 or the fluid flow through processing device 11 (which may affect the dissolution rate of tablet 18A) may affect the selection of tablet components.

Some tablet components may be more soluble or dissolve in a fluid, such as water, at a greater rate in certain fluids than others. In certain cases, a tablet component may be substantially insoluble in a fluid or highly soluble in another. Considerations such as the solubility of the tablet components and type of fluid flow through the particular processing device 11 may be taken into account when selecting tablet components. In some embodiments, selecting tablet components (22) may only include selecting one or more reagents, and the matrix material may be pre-selected or tablet 18A may not include a matrix material. In other embodiments, selecting tablet components (22) may include selecting one or multiple reagents and one or multiple matrix materials. Embodiments may further include selecting other tablet components, such as lubricating material, desired to be in tablet form.

For certain embodiments of the present inventions, tablet components may be prepared (24) before tablet is formed (26). The tablet components may be prepared for forming into tablet 18A prior to or after a user selects the tablet components (22). All tablet components may be prepared (24) in some embodiments while only a subset of components out of all tablet components may be prepared in other embodiments. In some embodiments, all tablet components are not prepared prior to forming of a tablet.

Preparation of tablet components (24) may include a variety of techniques. Such techniques include techniques known to those with skill in the art. Preparation by certain techniques may influence the properties of a tablet according to the present invention. In some embodiments, a tablet includes a substantially uniform distribution of reagent material and matrix material. Generally, substantially uniform distribution within a tablet can be promoted by preparation of tablet components prior the forming of a tablet. Tablet components may be prepared using techniques relating to dry chemical blends, such as, for example, lyophilizing technology, fluidized bed coating technology, dry mixing technology, and the like. Techniques such as these may produce a mixture of tablet components in which the components are substantially uniformly distributed in the mixture results in a substantially uniform distribution within a tablet.

Preparing tablet components by lyophilizing technology generally includes freeze drying the reagent and matrix material, as well as the other tablet components and dehydrating the tablet components, e.g., with the aid of a vacuum or a heat source. Each tablet component may be freeze dried together, or two or more tablet components may be lyophilized together. In one embodiment of preparing tablet components by fluidized bed coating technology, a reagent in an aqueous solution is sprayed onto a soluble or an insoluble fluidized bed, e.g., a microcrystalline cellulose bed. The reagent may be sprayed, e.g., via an atomizing sprayer or another type of spray that distribute the reagent solution in fine particles over the fluidized bed. The fluidized bed may be agitated during the reagent spraying process in order to encourage uniform distribution of the reagent on the bed. The fluidized bed coating technique may eliminate the need to lyophilize a reagent. The insoluble fluid bed is then dehydrated. After the insoluble material is compressed into a tablet, the reagent may be released by hydrating the insoluble material with a fluid, thereby causing the insoluble material to swell and release the reagent.

Preparing tablet components by dry mixing technology generally includes mixing the desired amounts of a dried reagent (e.g., in powder form) and dried forms of the other tablet components together in the desired ratios. The dry mixing technology may be useful for embodiments in which there is a greater tolerance for the amount of reagent that is within tablet 18A because, in some cases, the dry mixing may result in a non-uniform distribution of the reagent, particularly between tablets formed from the same batch of dry mixed tablet components. However, such differences in the amount of reagent between tablets 18 may be minimized by thorough mixing.

Dry mixing may be useful for certain reagents in which the tolerance to achieve a particular reaction is relatively large, such as reactions that require a relatively large range of reagent concentration window (e.g. a reagent concentration window ranging from about 30% to about 80% by tablet weight, such as about 50%) for a particular reaction to take place. For example, for some reagents used for sample preparation, e.g., a lysis reagent, the necessary concentration of the reagent within process chamber 14A to achieve the particular reaction is relatively large as compared to a reagent where the concentration windows are narrower. In reactions with relatively narrow reagent concentrations windows, e.g., a probe for PCR, it may be more important to have substantially homogenous composition of tablet components used to form a tablet because the concentration window for which a successful reaction will take place may only range from about 10% to about 20% by tablet weight, such as about 10%. If tablet components are not substantially homogenous in composition when formed into a tablet, concentration of the tablet components, e.g., a reagent, may vary between individual tablets. If there is too much variation, an individual tablet may not contain the tablet component concentration, e.g., concentration of a reagent, to successfully carry out a reaction.

Tablet 18A may be formed from tablet components (26). As shown, tablet 18A is formed from tablet components after tablet components have been selected and prepared, although not all embodiments include selection and preparation of tablet components. As discussed before, tablet components may include reagents, matrix materials, and lubricants. In some embodiments, a tablet may include at least one reagent and a matrix material. For example, a tablet may include a single reagent and a single matrix material. In some embodiments, a tablet may include a first reagent, a second reagent, and at least one matrix material. In still other embodiments, a tablet may contain a single reagent, a first matrix material and a second matrix material. In general, tablet according to the present invention may contain any permutation of tablet components. Multiple tablets may be formed in which some tablets have different tablet components than others. A first tablet including a first reagent and a first matrix material may be formed, and a second tablet including a second reagent and a second matrix material may also be formed, resulting in a first and second tablet with different compositions.

As discussed before, in some embodiments of a tablet, such as tablet 18A, may include an active component and a substantially solid reconstitution buffer. In general, a tablet including a substantially solid reconstitution buffer may be formed as described before. In such embodiments, a technique may include selecting an active component that requires reconstitution buffer prior to use in a chemical reaction, selecting a substantially solid reconstitution buffer and forming a tablet including the active component and the solid reconstitution buffer. In some embodiments, such a tablet is sized to fit within at least one chamber of a microfluidic device.

Forming tablet 18A may include those techniques generally known in the art for forming tablets. In some embodiments, forming a tablet includes compressing tablet components to define a tablet. Generally, compressing tablet components to form tablet 18A may include reducing the overall volume of tablet components, which are typically in powder form, by applying an elevated pressure to the components to define a tablet. In some embodiments, the compression pressure applied to form a tablet ranges from about 0.5 MPa to about 500 MPa, such as about 15 MPa to about 200 MPa or about 50 MPa to about 150 MPa. Some embodiments of the present invention may provide for compression of at least a reagent to form a tablet without significant deleterious results to the reagent(s), e.g., substantially no denaturization to an enzyme reagent.

A plurality of tablets 18 may be formed automatically or manually. In an automatic technique, a computing device may control at least one of the steps of tablet component preparation (24) or formation of the tablets 18. In a manual technique, an operator may control the preparation of tablet components (24) and formation of the tablets 18 alone or with the aid of a computing device. Automated techniques may be desired if relatively large quantities of tablets 18 are desired. For example, the operator may introduce a powder or otherwise solid form of the tablet components into a well of a tablet press, and manually apply compression pressure to compress the tablet components into a tablet form or manually active an automated device to apply the compression pressure.

Dimensions of tablet 18A are configured to fit within at least one process chamber 14A of processing device 11. In some embodiments, tablet 18A is generally sized to fit within chamber 14A such that the volume of tablet 18A is substantially enclosed within the volume define by chamber 14A of processing device 11. For example, tablet 18A may be configured to fit within process chamber 14A such that there is space between tablet 18A and sidewalls of process chamber 14A in order to permit fluid to contact at least a portion of the tablet 18A surface. That is, it may be desirable to size tablet 18A and process chamber 14A relative to each other such that a sufficient amount of fluid to at least partially dissolve tablet 18A may be disposed within process chamber 14A when tablet 18A is also present within chamber 14A. In addition, in some embodiments, in may be desirable for tablet 18A to be sized to fit within process chamber 14A such that sufficient surface area of tablet 18A is exposed to a fluid that is introduced into process chamber 14A in order to promote at least partial dissolution of tablet 18A, such that the reagent within tablet 18A may react with the fluid (e.g., the analyte).

Dimensions of tablet 18A according to the present invention are defined by the outer surface of tablet 18A, as well as the corresponding volume. A greatest dimension of tablet 18A may be the greatest dimension of a cross-section of tablet 18A or a dimension of a major surface of tablet 18A. For example, if tablet 18A is a cylindrical shape, the greatest dimension may be measured along the length of the cylinder or the greatest dimension may be a diameter of the cylinder. Tablet 18A may have any suitable dimensions in accordance with the present invention. For example, a tablet's dimensions may substantially define a cylinder, a rectangular prism or a triangular prism. In other embodiments, tablet dimensions may have a substantially asymmetrical, symmetrical or irregular shape.

In certain embodiments, a tablet may have dimensions such that the tablet may be considered a “microtablet.” For example, a substantially cylindrical tablet in which a major surface is about 5 millimeters (mm) or less, such as less than about 3 mm, may be considered a microtablet. In another example, a substantially cylindrical tablet with a circular face comprising a diameter of about 0.5 mm to about 3 mm may be considered a microtablet. In another example, a tablet with a volume ranging from about 1.0×10⁻² cubic millimeters (mm³) to about 100 mm³, such as about 5 mm³ or about 20 mm³, may be considered a microtablet. In some embodiments, a tablet may be considered a microtablet because of the tablet's weight. For example, a tablet with a weight ranging from about 50 milligrams to about 0.05 milligrams, such as about 30 milligrams to about 0.5 milligrams, may be considered a microtablet.

Micro tablets are not commonly manufactured because of the complexities involved in tooling manufacture and the inherent problems of powder trituration and flow. Furthermore, conventional belief was that it may be difficult to form a microtablet including a uniform distribution of the materials. However, it has been found that microtablets comprising a biological reagent for use in processing device 11 and a matrix material, such as a sugar, may be formed to have a substantially uniform distribution of the reagent and matrix material.

It has also been found that reagents in a microtablet form may dissolve faster than expected because the amount of matrix material within the microtablet is minimized. In some embodiments, the microtablet may include up to 95% of the reagent. A reagent material may be easier to compress into a microtablet because of the small volume and the relatively large surface area to volume ratio. Many factors, such as the relatively large surface area to volume ratio of microtablets, the minimization of bulking agents that may not be soluble, and the increase in soluble components, contribute to reagent microtablets exhibit a relatively fast dissolution rate within process chamber 14A of processing device 11. A relatively fast dissolution rate may be desirable in order to provide a processing assembly 10 that provides a relatively fast reaction time.

Forming tablet 18A (26) may include consideration of different parameters, such as the relative humidity of the operating environment of the tableting device (e.g., a tablet press) and the compression pressure applied to the tablet components by the tableting device to form the tablet. In some embodiments, one or more of the components to be formed into a tablet, such as, for example, a reagent or a matrix material, may be relatively hydrophilic. If the tablet components absorb water during a tablet formation process, the consistency of the tablet components may change, thereby potentially adversely affecting the consistency of tablet 18A (which may change the dissolution rate of tablet 18A within process chamber 14A) or even the ability to compress the components to form tablet 18A. In addition, if a substantially uniform distribution of a reagent and matrix material is desired, absorption of water from the operating environment may adversely affect any uniform distribution of the reagent and matrix material. Thus, in some embodiments, a low operating relative humidity prior to, during and after the formation of a tablet may be desirable. For example, in some embodiments, the relative humidity of the operating environment may be less than about 70%, such as less than about 50%, about 1% to about 30%, or about 5% to about 30%.

While processing assembly 10 including a processing device 11 with a single loading chamber 14 fluidically coupled to multiple process chambers 14 is described above, in other embodiments, other types of processing devices that include at least one reagent may include a tablet comprising the reagent. For example, the processing devices similar to those described in the following patents and patent applications may include a reagent in a tablet form: U.S. Patent Application Publication Nos. 2005/0129583 (Bedingham et al.); 2007/0009391 (Bedingham et al.); as well as U.S. Pat. Nos. 6,627,159 (Bedingham et al.), 6,734,401 (Bedingham et al.), 6,987,253 B2 (Bedingham et al.), 6,814,935 (Harms et al.), 7,026,168 (Bedingham et al.), 7,192,560 (Parthasarathy et al.), and 7,322,254 (Bedingham et al.), which are each incorporated herein by reference in their entireties. The documents identified above all disclose a variety of different constructions of processing devices that may include a tablet comprising a reagent. The devices may preferably include fluid features designed to process discrete microfluidic volumes of fluids, e.g., volumes of 1 milliliter or less, 100 microliters or less, or even 10 microliters or less.

In addition, while processing device 11 including a single supply input chamber 12 is primarily described above, in other embodiments, a processing device including a plurality of supply input chambers may include a tablet comprising a reagent. FIG. 3 is a schematic diagram of an embodiment of another exemplary processing assembly 30 that may include a plurality of reagent tablets according to the present invention. Similar to FIG. 1, assembly 30 includes processing device 31. Processing device 31 includes a plurality of process chambers 34, a plurality of loading chambers 32 and a plurality of conduits 36. Each process chamber 34 is fluidically coupled to a loading chamber 32 by conduit 36, respectively. As such, loading chamber 32 may supply a fluid (e.g., a sample material, a buffer, or the like) to conduit 36 and process chamber 34 of device 31. Only the portion of each conduit 36 that connects to process chamber 34 is illustrated. A tablet 38 is disposed in each of process chambers 34, but is not illustrated in FIG. 3. In the embodiment shown in FIG. 3, conduits 36 are each a microfluidic conduit. Thus, processing device 31 may also be referred to as a “microfluidic processing device.” Assembly 30 functions substantially similar to FIG. 1 as described before.

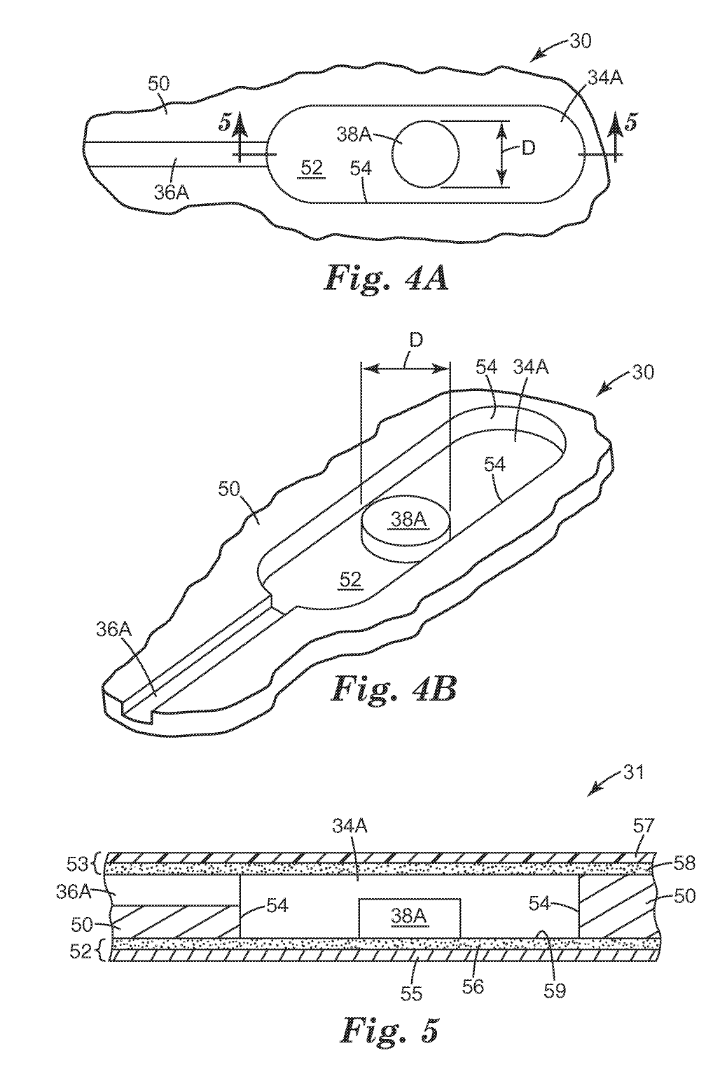

FIGS. 4A and 4B are magnified views illustrating process chamber 34A and conduit 36A of the exemplary processing assembly 30 of FIG. 3. Conduit 36A connects process chamber 34A to a respective loading chamber 32A (shown in FIG. 3) such that fluid, e.g. analyte, from loading chamber 32A may be supplied to process chamber 34A through conduit 36A. Tablet 38A has been configured to fit within process chamber 34A, which is defined by first layer 52 of processing device 31, sidewalls 54, and a second layer 53 (not shown in FIG. 4) of processing device 31. As such, tablet 38A is sized to fit within process chamber 34A. The volume of space in chamber 34A between tablet 38A and first layer 52, sidewalls 54 and/or second layer 53 is sufficient to allow for fluid to contact at least a portion of tablet 38A. As shown in FIG. 4, tablet 38A comprises a substantially cylindrical shape including a circular cross-section. In embodiments in which tablet 38 is a microtablet with a substantially cylindrical shape, diameter D of the cross-section of tablet 38A may be less than about 5 mm, such as less than about 3 mm.

FIG. 5 is a partial cross-sectional view of process chamber 34A and conduit 36A of FIGS. 4A and 4B taken along line 5-5 in FIG. 4A, and illustrates tablet 38A disposed within process chamber 34A. In the embodiment shown in FIG. 5, processing device 31 of processing assembly 30 is comprised of multiple layers, including a substrate 50, a first layer 52, and a second layer 53. Substrate 50, first layer 52, and second layer 53 are preferably bonded or attached together to contain a fluid (e.g., an aqueous fluid) without leakage of the fluid through the bond or attachment between substrate 50 and first layer 52 or second layer 53. The bond or attachment may be, for example, a pressure sensitive adhesive, ultrasonic welding, hot melt adhesive, thermoset adhesive, a thermal bond or static charge. The type of bond or attachment may be selected based on the anticipated conditions for using tablet 38. For example, a pressure sensitive adhesive may be selected if tablet 38 is to be used in an aqueous environment. In the embodiment shown in FIG. 5, optional bonding layer 56 may bond first layer 52 to substrate 50, and optional bonding layer 58 may bond second layer 53 to substrate 50.

Chamber 34A of device 31 is in fluid communication with conduit 36A, which is also in fluid communication with a respective loading chamber 32A (not shown). As previously described, loading chamber 32A may supply a fluid (e.g., an analyte, a buffer, or the like) to conduit 36A and chambers 34A of device 31. In the embodiment shown in FIG. 5, conduit 36A is formed in substrate 50 and enclosed by second layer 53. In other embodiments, conduit 36A may be on an opposite side of substrate 50 enclosed by first layer 52.

First layer 52 includes support layer 55 and second layer 53 includes support layer 57. Support layers 55 and 57 can each be comprised of one layer or multiple layers, can be a polymeric film such as described herein for the support film, can be a metallic layer, or a combination of a polymeric film and a metallic layer. Support layers 55 and 57 may or may not be the same. When support layers 55 and/or 57 are metallic, the respective optional bonding layers 56, 58 may be present to separate process chamber 34A from the metal of the metallic layer. In embodiments in which detection is made via fluorescence detection or color change detection within process chamber 34A, it may be desirable for at least one of support layers 55 and 57 to be formed from a nonmetallic layer in order to provide the capability of detecting fluorescence through the respective layer 55 and 57.

In FIG. 5, tablet 38A is disposed within process chamber 34A such that tablet contacts first layer 52. In other embodiments, tablet 38A may be disposed within process chamber 34A so as contact any one of the walls of the chamber 34A, including second layer 53 or sidewalls 54. For example, tablet 38A may be adhered to surface of first layer 52 of process chamber 34A by bonding layer 56 which may be an adhesive layer that is configured to adhere tablet 38A to first layer 52. In yet another embodiment, optional bonding layer 58 may adhere tablet 38A to second layer 53. Optional bonding layers 56 and 58 may be any suitable bonding material, such as a pressure sensitive adhesive, hot melt adhesive, thermoset adhesive, other adhesives or other thermal bonds.

Tablet 38A may be adhered to bottom surface 59 of process chamber 34A by an optional pressure sensitive adhesive layer that is applied to tablet 38A and/or bottom surface 59 of process chamber 34A. Instead of or in addition to optional adhesive layer, bonding layer 56 may be an adhesive layer that is configured to adhere tablet 38A to first layer 52 of device 31. In yet another embodiment, optional bonding layer 58 may adhere tablet 38A to second layer 54 instead of or in addition to a separate adhesive layer. Optional bonding layers 56 and 58 and optional adhesive layer 60 may be any suitable bonding material, such as a pressure sensitive adhesive, hot melt adhesive, thermoset adhesive, other adhesives or other thermal bonds.

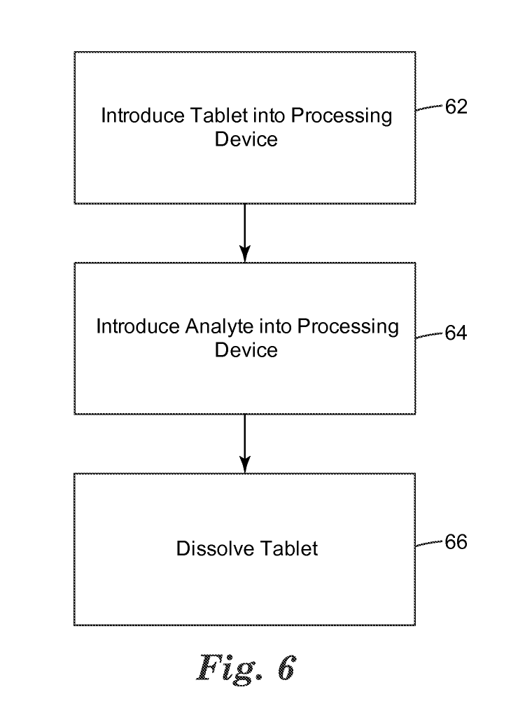

FIG. 6 is a flow chart illustrating an exemplary technique for using a tablet in a processing device according to the present invention. While processing assembly 30 including device 31 and tablets 38 are primarily referred to throughout the description of FIG. 6, in other embodiments, the technique shown in FIG. 6 may be used with other types of processing devices and tablets. In some embodiments, such as that illustrated in FIGS. 3-5, tablet 38A has been introduced into process chamber 34A of processing device 31A such that tablet 38A is disposed entirely within chamber 34A (62). In one embodiment, tablet 38A is introduced into chamber 34A with the aid of a computer-controlled apparatus (e.g., a robotic arm), as described in U.S. Provisional Patent Application No. 60/985,827, entitled “CHEMICAL COMPONENT AND PROCESSING DEVICE ASSEMBLY,” filed on Nov. 6, 2007.

Tablet 38A may be introduced into processing chamber 34A (62) to help aid a particular sample processing or detection technique. Typically, tablet 38A is introduced into chambers or other regions of processing device 31 that may require a chemical to aid a particular reaction. For example, tablet 38A may be disposed within process chamber 34A because it may be desired for a chemical reaction associated with a processing technique to take place substantially within the boundaries of chamber 34A. Other embodiments may exist in which a tablet is disposed at a location within processing device 31 and then transferred to another location within device 31 (e.g., another process chamber) before, during or after processing of a sample, such as an analyte, within the device.

In accordance with the technique shown in FIG. 6, an analyte may be introduced into a processing device (64), e.g. via loading chamber 32. For example, a user may pipette a controlled amount of the analyte into loading chamber 32A, where the controlled amount is selected to accommodate the particular processing device 31, which may contain a limited volume of fluids. In processing device 31, loading chamber 32A is coupled to a respective process chamber 34A. Thus, by loading the analyte into loading chamber 32A, the analyte may be moved into process chamber 34A via any suitable technique, e.g., via a centrifugal force, vacuum force, gravitational force, and so forth. In other embodiments, such as processing device 11 in FIG. 1, two or more process chambers may be fluidically coupled to a common loading chamber.

In certain embodiments, at least a portion of a fluid, such as an analyte, that has been introduced into a loading chamber (64) is introduced to any or all of the process chambers via corresponding conduits, as described above. In embodiments in which a tablet according to the present invention has been previously introduced into the respective process chamber, the introduction of at least a portion of a fluid, e.g., an analyte, into a process chamber typically brings about an interaction, such as fluid mixing, between the tablet and the fluid. In some embodiments, interaction between a tablet and fluid will dissolve, disperse or suspend tablet components within a processing chamber. In certain embodiments, fluid may at least partially dissolve a tablet within the process chamber (66) and/or may react with at least some tablet components. For example, an analyte may dissolve some tablet components and also chemically react with some tablet component, such as a reagent, or more generally, an active tablet component.

In general, depending on the composition of tablet 38A, tablet 38A may at least partially dissolve within processing device 31, specifically when interacting with a fluid. Process chamber 34A may be configured to help aid dissolution of tablet 38A. For example, process chamber 34A may include curvilinear side walls 54 and first and second layers 52, 53 may include curved surfaces near process chamber 34A to encourage the flow and speed of fluid flow through process chamber 34A. Furthermore, the flow of fluid around process chamber 34A and around tablet 38A may further be aided with other fluid control techniques, such as by applying a vacuum force that causes fluid to flow in and out of chamber 34A or causes tidaling of fluid within chamber 34A, by rotating device 31 in a particular pattern (e.g., patterns involving acceleration and deceleration of device 31 rotation, as well as changing the direction of rotation), and so forth. In addition, an operator or a computer-controlled device may manually manipulate (e.g., shake) device 31 to encourage flow around tablet 38A.

When at least partially dissolved, the reagent from tablet 38A may be at least partially suspended in fluid. In some embodiments, all matrix material components of tablet 38 may be substantially dissolved in an analyte within a process chamber. In some embodiments, tablet 38A comprises a composition that permits tablet 38A to substantially dissolve in process chamber 34A within about 600 seconds from the introduction of a fluid, e.g. an analyte, into the chamber. In certain embodiments, tablet 38A substantially dissolves in process chamber 34A within about 3 second to about 300 seconds from the introduction of a fluid into chamber 34A, such as about 30 to about 180 seconds from the introduction of a fluid into the chamber. Tablet components can dictate the dissolution rate of a tablet in an analyte and may be selected in a manner to control or influence the dissolution rate of a tablet in an analyte with a process device. For example, tablet 38A including sorbitol as a matrix material may dissolve faster than a tablet that uses maltose as the matrix material. In another embodiment, tablet 38A may include a substantially water-insoluble lubricant to decrease the dissolution rate of tablet 38A or may include a disintegrant to increase the dissolution rate of tablet 38A.

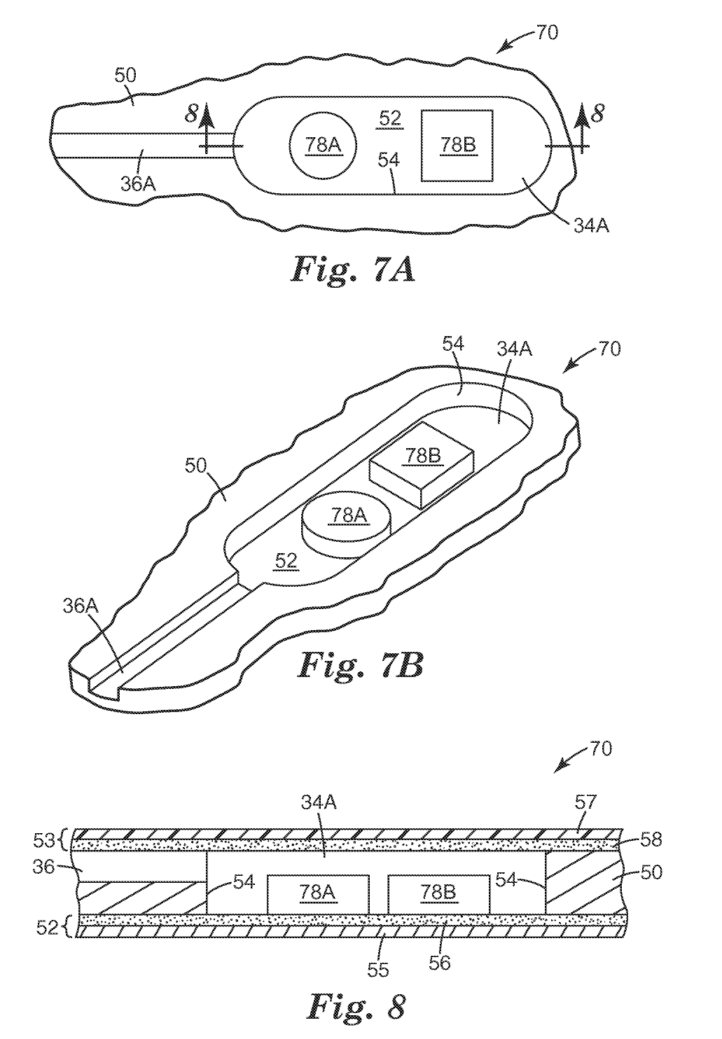

In some embodiments, a processing device may include more than one tablet within a single process chamber. FIGS. 7A and 7B are schematic top views of a process chamber 34A of an exemplary processing assembly 70. Processing assembly 70 is substantially similar to processing assembly 30 of FIG. 3 as described above except processing assembly 70 includes first tablet 78A and second tablet 78B disposed within process chamber 34A. First tablet 78A and second tablet 78B are configured such that both tablets 78A and 78B fit within process chamber 34 as defined by first layer 52, sidewalls 54, and second layer 53 (not shown). Tablet 78A and tablet 78B are sized such that a volume of chamber 34 remains unoccupied by tablets 78A and 78B to allow a fluid to contact at least a portion of the surface of tablets 78A and 78B and at least partially dissolve tablets 78A and 78B. As shown in FIG. 7A, first tablet 78A has a substantially oval cross-section (taken along the plane of the image in FIG. 7A), and second tablet 78B has a substantially square cross-section (taken along the plane of the image in FIG. 7A).

FIG. 8 is a partial cross sectional view of process chamber 34A and conduit 36A of FIGS. 7A and 7B taken along line 7-7 in FIG. 7A, and illustrates first tablet 78A and second tablet 78B disposed within process chamber 34A. First tablet 78A and second tablet 78B are in contact with first layer 52.

In some cases in which multiple tablets (e.g., tablets 78A, 78B) are in a single process chamber 34A of processing device 31, a composition of two or more of the tablets may vary. For example, in one embodiment, tablet 78A may have a different composition than tablet 78B. As nonlimiting examples of different tablet compositions, tablet 78A may include a first reagent and tablet 78B may contain a second reagent that is different than the second reagent. In other embodiments, a first tablet may contain the same reagent as a second tablet, but the first tablet may contain a first matrix material and the second tablet may contain a second matrix material different from the first matrix material.

Tablets 78A, 78B with different compositions, such as those embodiments described above, may differ in dissolution rates when brought into contact with a fluid within process chamber 34A. For example, in one embodiment, tablet 78A may completely dissolve in less than a minute in the presence of an analyte, while tablet 78B may not begin dissolving until after tablet 78A dissolves, where tablet 78B completely dissolves in about 120 to about 180 seconds after the analyte contacts tablets 78A, 78B. Tablets 78A, 78B may be engineered to at least partially dissolve at different rates, e.g., by varying the type or quantity of matrix material. Tablets 78A, 78B with different dissolution rates may be useful if a reaction involving two different reagents takes place within a common process chamber 34A, and it is desirable for the reagents to react with the analyte at different times.

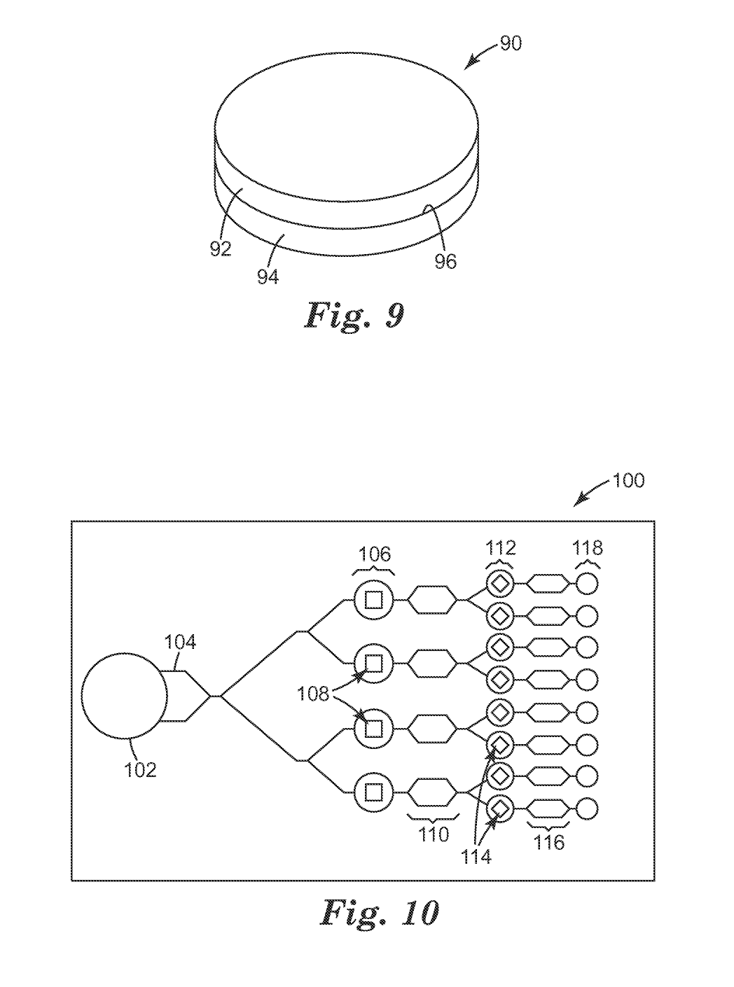

In some embodiments, a tablet may include two or more reagents. FIG. 9 illustrates an exemplary tablet 90 according to the present invention comprising a first layer 92 and a second layer 94. As shown, first layer 92 is substantially distinct from second layer 94 and is separated from second layer 94 at interface 96. The composition of first layer 92 may be difference from the composition of second layer 94. For example, first layer 92 may include a first reagent and a first matrix material, and second layer 94 may contain a second reagent and a second matrix material, in which the first reagent and first matrix material are different from the second reagent and second matrix material, respectively. In some embodiments, first layer 92 may have reagent(s) that are different from the reagent(s) in second layer 94 but include the same matrix material. Conversely, first layer 92 may have matrix material(s) that are different from the matrix material(s) in second layer 94 but include the same reagent material. The tablet components of layers 92 and 94 may be selected such that layers 92 and 94 have different dissolution rates in the presence of a fluid.

In general, a tablet according to the present invention including multiple layers of different compositions may allow for flexibility in processing techniques. For example, tablet layer compositions may be configured such that layer 92 of tablet 90 dissolves at a faster rate than layer 94 after being brought into contact with a fluid within a processing chamber. As a result, components of layers 92, 94 of tablet 90 may react with an analyte or be released within a process chamber at different times. In general, first layer 92 may be substantially uniform in composition and second layer 94 may be substantially uniform in composition. Although tablet 90 has only two layers, other embodiments may include two or more layers having different compositions. Furthermore, tablet 90 may include different reagents in arrangements other than distinct layers, e.g., a swirling formation in which two different reagents and/or matrix portions of tablet 90 are distinct portions.

A tablet with different reagents, e.g., tablet 90 of FIG. 9, may be formed by one or more techniques, including techniques known in the art of tableting and the like. For example, first layer 92 having a first composition and second layer 94 having a second composition may be formed individually. First layer 92 may be formed by compressing tablet components having a first composition as described above. Second layer 94 may be formed using the same technique as used to form the first layer 92. First layer 92 and second layer may then be coupled to each other, for example, at interface 96 as illustrated in FIG. 9.

Other technique may be used to form a tablet with multiple layers, e.g., tablet 90 of FIG. 9. For example, a first composition and second composition may be disposed such that two substantially distinct composition layers are formed prior to forming tablet 90. In some embodiments, a first composition is prepared such that it is substantially uniform in composition and a second composition is prepared such that it is substantially uniform in composition. Tablet 90 is formed, for example, by compressing the first composition and a second composition together to form tablet 90 with a first layer 92 and second layer 94 corresponding to the first composition and second composition, respectively. In some embodiments, a first composition and second composition, both in powder form, may be dispensed sequentially into a tablet die cavity and then compressed to form tablet 90 with multiple substantially distinct layers of either first or second composition. Once again, although tablet 90 has two layers, other embodiments may include forming a tablet having two or more layers having different compositions using techniques such as those described herein.

While both processing devices 11 (FIG. 1) and 31 (FIG. 3) have a single “tier” of process chambers 14, 34 such that fluid does not flow past each process chamber 14, 34 or substantially all reactions take place within a single process chamber 14, 34, in other embodiments, a chemical component may be placed within a processing device that includes two or more process chambers provided in a sequential relationship. The process chambers may be separated by a fluid control structure, such as a laser valve or another type of valve. FIG. 10 is a schematic illustration of processing device 100, which includes multiple process chambers in a sequential relationship. While one set of process chambers is shown in FIG. 10, in other embodiments, a plurality of sets of process chambers arranged similarly to that shown in FIG. 10 may be repeated about a common axis, as with processing device 11 and process chambers 14. An example of processing device 100 that includes fluid structures with multiple, connected process chambers is described in U.S. Pat. No. 6,734,401, entitled “ENHANCED SAMPLE PROCESSING DEVICES SYSTEMS AND METHODS,” (Bedingham et al.), which is incorporated herein by reference in its entirety.

As shown in FIG. 10, a loading chamber 102 is provided to receive, e.g., a starting sample material. The array and one illustrative method of using the array will be described below. The illustrative method involves PCR amplification, followed by Sanger sequencing to obtain a desired end product. This combination of processes is, however, intended to be illustrative only and should not be construed as limiting the types of processing devices in which a chemical component may be placed in accordance with the techniques and systems described herein.

In one example, a starting sample material, such as lysed blood cells, is provided in loading chamber 102. Filter 104 may be provided to filter the starting sample material as it moves from the loading chamber 102 to first tier of process chambers 106. Filter 104 is, however, optional and may not be required depending on the properties of the starting sample material. In one embodiment, first process chambers 106 includes tablet 108, which includes a suitable PCR primers. Each of first process chambers 106 may include tablet 108 or a tablet with a different composition, depending on the nature of the investigation being performed on the starting sample material. One alternative to providing the primers in first process chambers 106 before loading the sample is to add a suitable primer to the loading chamber 102 with the starting sample material (provided that the primer is capable of passing through the filter 104, if present). In FIG. 10, as well as the other figures of the disclosure, the tablets are not shown to scale relative to the process chambers.

After locating the starting sample material and any required primers in first process chambers 106 and dissolving tablet 108, the materials in first process chambers 106 are thermally cycled under conditions suitable for PCR amplification of the selected genetic material. After completion of the PCR amplification process, the materials in each of first process chambers 106 may be moved through filter chamber 110 to remove unwanted materials from the amplified materials, e.g., PCR primers, unwanted materials in the starting sample that were not removed by filter 110, etc. In the embodiment shown in FIG. 10, each process chamber 106 is fluidically coupled to one filter chamber 110. The filter chambers 110 may, for example, contain size exclusion substances, such as permeation gels, beads, etc. (e.g., MicroSpin or Sephadex available from Amersham Pharmacia Biotech AB, Uppsala, Sweden).

After clean-up of the sample materials in filter chambers 110, the filtered PCR amplification products from each of the first process chambers 106 are moved into a pair of multiplexed second process chambers 112 for, e.g., Sanger sequencing of the genetic materials amplified in the first process chambers 106 through appropriate control of the thermal conditions encountered in second process chambers 112. Disposed within each of second process chambers 112 is a tablet 114 containing a component, which may be used for Sanger sequencing. Tablets 114 may be placed within each of second process chambers 112 prior to, during or after tablets 108 are placed within first process chambers 106.

After the desired processing has been performed in second process chambers 112, the processed material (Sanger sequenced sample material if that is the process performed in second process chambers 112) is moved from each of second process chambers 112 through another set of filter chambers 116 to remove, e.g., dyes or other unwanted materials from the product of second process chambers 112. The filtered product is then moved from the filter chambers 116 into output chambers 118, where the product may be removed.

Chambers 102, 106, 112, and 118 may be arranged generally radially on device 100 such that rotation of device 100 will move materials from the loading chamber 102 towards the output chambers 118. For example, two or more of the process chamber arrays illustrated in FIG. 10 may be arranged on a single device, with the loading chambers 102 of each array located closest to the axis of rotation such that the materials can be moved through the array by centrifugal forces developed during rotation. Alternatively, the arrays may be located on a device that is held in a manner that allows rotation of device containing the array such that centrifugal forces move the materials from the loading chamber 102 towards the output chambers 118. Loading of sample materials into process chambers using centrifugal force is also described, for example, in U.S. Pat. No. 6,627,159, entitled, “CENTRIFUGAL FILLING OF SAMPLE PROCESSING DEVICES” (Bedingham et al.).

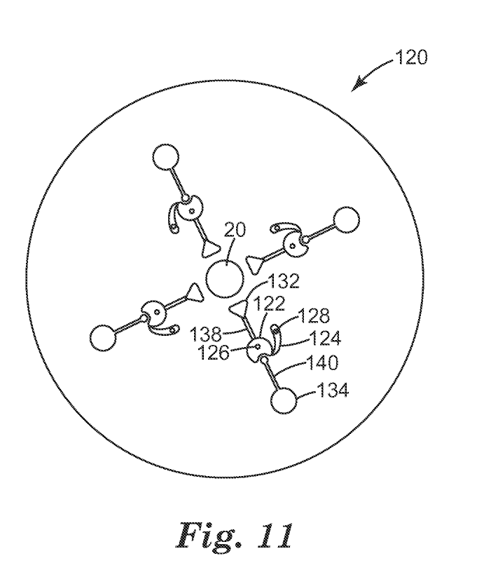

In other embodiments, a processing device may include a process chamber that is fluidically coupled to mixing chambers that aid a mixing process. In such embodiments of processing devices, reagent tablets may be disposed within the process chamber and mixing chambers, where the tablets may be the same or different. FIG. 11 is a schematic top view of an exemplary processing device 120, which includes process chamber 122 fluidically coupled to mixing chamber 124. Process chamber 122 is also fluidically coupled to loading chamber 132 by conduit 138. Output process chamber 134 is fluidically couple to process chamber 122 by conduit 140. Loading chamber 132 may supply a fluid (e.g., a sample material, a buffer or the like) to process chamber 122 via conduit 138.

Tablet 126 is disposed within process chamber 122 and tablet 128 is disposed within mixing chamber 124. As fluid flows between process chamber 122 and mixing chamber 124, tablets 126 and 128 may dissolve. In general, as process device 120 rotates about central axis 20, fluid in process chamber 122 enters mixing chamber 124 at least partially because of centrifugal forces generated by the rotation of device 120. Fluid entering mixing chamber 124 compresses the volume of the gas in chamber 124, increasing the gas (e.g., air) pressure within chamber 124. As the rotation of device 120 decreases in speed or ceases, the centrifugal force within chambers 122, 124 decreases and the gas within mixing chamber 124 forces some or all of fluid in mixing chamber 124 back into process chamber 122. That is, after rotationally accelerating device 120, gas within mixing chamber 124 may become at least partially elevated in pressure due in part to the introduction of fluid from process chamber 122 to mixing chamber 124. Such a technique of accelerating and decelerating the rotation of device 120 around center axis 20 may be used to increase the rate of dissolution of tablets 126, 128 by encouraging the mixing of fluid with tablets 126 and 128 within the respective chambers 122, 124.

In some embodiments, tablet 126 is first dissolved in process chamber 122 because fluid is first introduced into process chamber 122 from loading chamber 132. As device 120 is rotated, fluid flows into mixing chamber 124, thereby dissolving tablet 128. In some embodiments, one or more tablets may be disposed within process chamber 122 and/or mixing chamber 124, while in other embodiments, at least one of process chamber 122 or mixing chamber 124 may not include a tablet. In some embodiments, process device may have more than one mixing chamber.

### EXAMPLES

Objects and advantages of this invention are further illustrated by the following examples, but the particular materials and amounts thereof recited in these examples, as well as other conditions and details, should not be construed to unduly limit this invention.

### Example 1

Microtablets were formed from a mixture including lysostaphin and sorbitol using a hand operated Arbor Press. Lysostaphin powder (CAS No. 9011-93-2; L4402 Lysostaphin from Staphylococcus staphylolyticus) at approximately 50% protein by biuret was obtained from Sigma Chemicals, St. Louis, Mo. The lysostaphin powder was triturated using an agate mortar and pestle. The lysostaphin powder then mixed with 25 parts by weight sorbitol (NeoSorb® P60W), which was obtained from Roquette America, Inc., Keokuk, Iowa. The lysostaphin/sorbitol mixture was then vortexed on a Vortex-Genie (Fisher Scientific, Bohemia, N.Y.) to provide a well mixed powder exhibiting a substantially uniform distribution of the reagent, in this example, lysostaphin, and the matrix material, in this example, sorbitol.

The resulting mixture was formed into microtablets using a single leverage lab Arbor Press (Dake, Grand Haven, Mich.) fitted with a custom-made 1.2 mm diameter stainless steel punch and die set equipped with spacers for adjusting fill volume. The Arbor Press was operated using an electronic torque wrench (Model#7767A12 from Mc-Master Carr, Atlanta, Ga.). The fill volume was adjusted to obtain a compressed microtablet weight of 750 micrograms. Each microtablet contained about 15 micrograms of lysostaphin. The microtablets were compressed at a pressure of 155 MPa.

### Example 2

In the second example, it was determined that lysostaphin in a microtablet form (i.e., formed via the technique described above with respect to Example 1), exhibited substantially the same properties in a reaction to detect Methicillin-sensitive Staphylococcus aureus (MSSA). In particular, ATCC strain #25923 of MSSA (American Type Culture Collection; Manassas, Va.) was lysed using lysostaphin in solution form and lysed using lysostaphin in microtablet form, and the results were compared.

Specifically, MSSA bacteria were grown overnight through inoculation into Trypticase Soy Broth (TSB broth) and incubation at about 37° C. for approximately 18 hours. This overnight culture was then diluted from 1.4×10⁹ colony forming units (cfu)/milliliters (mL) to 1.4×10⁸ cfu/mL in 10 millimols (mM) Tris-HCl, 1 mM ethylenediamine tetraacetic acid (EDTA) (pH 8.0)/0.2% Pluronic® L64 (BASF; Mount Olive, N.J.) (hereinafter “TEP buffer”).

For first samples, (MSSA lysed using lysostaphin in solution form), about 200 μL of bacterial dilution containing 2.7×10⁷ cfu MSSA was mixed with about 60 μL of 10 mM Tris-HCl, 1 mM EDTA (pH 8.0) (hereinafter “TE buffer”) containing about 15 micrograms (μg) of lysostaphin (Sigma-Aldrich, St. Louis, Mo.). For second samples, (MSSA lysed using lysostaphin in microtablet form), about 200 μL of bacterial dilution containing 2.7×10⁷ cfu MSSA was mixed with about 60 μL of TE buffer with a dry lysostaphin microtablet containing about 15 μg lysostaphin from Example 1. Both the first and second samples were then gently vortexed and incubated at room temperature for about 10 minutes.

To obtain MSSA only control samples, about 200 μL of bacterial dilution containing 2.7×10⁷ cfu MSSA was mixed with about 60 μL of TEP buffer. To obtain first lysostaphin only control samples (solution form), about 200 μL of TEP buffer were mixed with about 60 μl, of TE buffer containing about 15 μg of lysostaphin. To obtain the second lysostaphin only control samples (tablet form), about 200 μL of TEP buffer were mixed with about 60 μL of TE buffer with a dry lysostaphin microtablet containing about 15 μg lysostaphin.

The first samples, second samples, and control samples were then serially diluted from 1.1×10⁸ cfu/mL to 1.1×10³ cfu/mL in TEP buffer. The first samples, second samples and MSSA only control samples were then quantified via blood agar plating. For the first and second lysostaphin only control samples, about 10 μL of bacterial dilution containing 1.4×10³ cfu was also added to the samples. These mixtures were then gently vortexed and incubated at room temperature for about 10 minutes. The first and second lysostaphin only control samples were then quantified via blood agar plating. Blood agar plating of all samples involved spreading about 200 μL solution onto blood agar plates, incubation at 37° C. for 16 hours, and subsequent enumeration of colony-forming units.

Table 1 shows the associated plate count data for MSSA bacteria kill. The plate count data in Table 1 demonstrates that in the second example, the second sample, i.e. the sample containing lysostaphin in microtablet form, killed substantially all MSSA bacteria, as the plate counts reflect no growth. Similarly, the first sample, i.e., the sample containing lysostaphin in solution form killed substantially all MSSA bacteria. Thus, the lysostaphin tablet provides substantially similar reagent properties as the lysostaphin liquids solution, but provides the additional advantage of easier handling and assembly with a processing device due to, at least in part, its mechanical and dimensional stability.

Additionally, residual lysostaphin after dilution (from solution form or tablet form) did not inhibit MSSA bacteria growth, as total counts of first and second lysostaphin control samples were comparable to the MSSA only control.

TABLE 1
Plate count data for MSSA bacteria kill.

| | | | Sample | Plate | Plate Count | | | Avg Plate | Total Count |
|---|---|---|---|---|---|---|---|---|---|
| Sample | | | (uL) | (uL) | (cfu) | | | Count (cfu) | (cfu) |
| Test | Lysostaphin Solution | | 1000 | 200 | 0 | 0 | 0 | 0 | 0 |
| | Lysostaphin | #1 | 1000 | 200 | 0 | 0 | 0 | 0 | 0 |
| | Tablet | #2 | 1000 | 200 | 0 | 0 | 0 | 0 | 0 |

| | | | | | | | | |
|---|---|---|---|---|---|---|---|---|
| Control | MSSA only | 1000 | 200 | 266 | 279 | 278 | 274 | 1372 |
| | Lysostaphin Solution | 1010 | 200 | 387 | 295 | 435 | 372 | 1880 |
| | Only | | | | | | | |
| | Lysostaphin Microtablet | 1010 | 200 | 301 | 273 | 289 | 288 | 1453 |
| | Only | | | | | | | |

### Example 3

In a third example, MSSA (as described in Example 2) DNA was extracted using lysostaphin microtablet (lysis), proteinase K solution (degradation), and heat (denaturation) for downstream quantitation by SAfemA-FAM real-time PCR (described below).

MSSA was grown overnight, as in Example 2, and then serially diluted to 1.4×10⁶ cfu/mL in TEP buffer. For MSSA DNA extraction, about 50 μL of bacterial dilution samples containing either 6.9×10³ cfu MSSA, 6.9×10² cfu MSSA, 6.9×10 cfu MSSA, or 0 cfu MSSA were mixed with about 60 μL of TE buffer containing about 15 μg lysostaphin or about 60 μL of TE buffer with a dry lysostaphin tablet containing about 15 μg lysostaphin from Example 1. The samples were then gently vortexed and incubated at room temperature for about 10 minutes. Next, about 15 μL of 20 mg/mL proteinase K solution (Qiagen, Valencia, Calif.) was added to each sample and the mixture was gently vortexed. Finally, the samples were heated at about 65° C. for about 10 minutes, then at about 95° C. for about 10 minutes, then cooled and stored on ice before real-time PCR.

Five microliters of each sample was subjected to real-time PCR amplification for femA gene from S. aureus (SA-femA) using a literature-reported method (International Publication No. WO 2002/082086 (A2, A3) (Schrenzel et. al)) with some further optimization regarding buffer, primer and probe concentrations as described below. The forward and reverse SA-femA primer sequences were TGC CTT TAC AGA TAG CAT GCC A and AGT AAG TAA GCA AGC TGC AAT GAC C, respectively. The SA-femA probe sequence was TCA TTT CAC GCA AAC TGT TGG CCA CTA TG labeled by fluorescein (FAM). PCR amplification was performed in an approximately 10 μL volume. The approximately 10 μL volume contained about 5 μL of the respective sample and about 5 μL mixture of forward primer and reverse primer (about 0.5 μL of about 10 μM of each primer); a probe (about 1 μL of about 2 μM probe); MgCl₂ (about 2 μL of about 25 mM MgCl₂); and LightCycler DNA Master Hybridization Probes master mix (about 1 μL of about 10× master mix) (available from Roche, Indianapolis, Ind.). PCR amplification was performed on the LightCycler 2.0 Real-Time PCR System (available from Roche, Indianapolis, Ind.) with the following protocol: about 95° C. for about 30 seconds (denaturation); 45 PCR cycles at about 95° C. for about 1 second (20° C./s slope), about 60° C. for about 20 seconds (20° C./s slope, single acquisition).

Table 2 shows the SAfemA-FAM PCR quantitative analysis data. The threshold cycle (Ct) results from Lysostaphin Tablet samples show a minimal 1-2 Ct shift as compared to respective Lysostaphin Solution samples. Also, Ct results for the overall dilution series from Lysostaphin Tablet samples show a similar trend as compared to Lysostaphin Solution samples. Thus, the results of Example 3 demonstrate that the lysostaphin in tablet form provides substantially similar reagent properties as the lysostaphin in liquid form.

TABLE 2
MSSA DNA Detection Results

| MSSA (cfu) | Lysostaphin | cfu/rxn | Ct | Avg Ct | Stdev Ct |
|---|---|---|---|---|---|
| 6.9E+03 | In solution | 270 | 27.96 | 27.90 | 0.05 |
| | form | | 27.87 | | |
| | | | 27.87 | | |
| 6.9E+02 | | 27 | 30.58 | 30.84 | 0.23 |
| | | | 31.00 | | |
| | | | 30.94 | | |
| 6.9E+01 | | 2.7 | 33.74 | 33.92 | 0.20 |
| | | | 34.13 | | |
| | | | 33.90 | | |
| 0 | | 0 | neg | neg | n/a |
| | | | neg | | |
| | | | neg | | |
| 6.9E+03 | In tablet | 270 | 29.62 | 29.53 | 0.09 |
| | form | | 29.45 | | |
| | | | 29.52 | | |
| 6.9E+02 | | 27 | 32.87 | 32.67 | 0.40 |
| | | | 32.21 | | |
| | | | 32.93 | | |
| 6.9E+01 | | 2.7 | 35.84 | 35.38 | 0.66 |
| | | | 34.91 | | |
| | | | neg | | |
| 0 | | 0 | neg | neg | n/a |
| | | | neg | | |
| | | | neg | | |
| TEP | n/a | n/a | neg | neg | n/a |
| TE | | | neg | neg | n/a |

### Example 4

Example 4 example demonstrates one use of a microtablets containing lysostaphin in a microfluidic device.

In the fourth example, three tablets comprising a greatest dimension of about 1.2 mm and including approximately 15 μg of lysostaphin from Example 1 were placed into three separate amplification and detection wells of a Fastman sample processing device, available from 3M Company of St. Paul, Minn. Different constructions of Fastman sample processing devices are described in, for example, U.S. Pat. Nos. 7,026,168 (Bedingham et al.); 6,814,935 (Harms et al.); 6,734,401 (Bedingham et al.); 7,192,560 (Parthasarathy et al.); 6,627,159 B1 (Bedingham et al.), and International Publication No. WO 2005/061084 A1 (Bedingham et al.). An 18-hour overnight culture of Staph aureus (MSSA, ATCC 25933) was diluted to 3.6×10⁶ colony forming units (cfus) per milliliter, 3.6×10⁵ cfu/mL, and 3.6×10⁴ cfu/mL, respectively, in approximately 10 mM Tris-HCl, approximately 1 mM EDTA (TE Buffer, supplied as a 10× solution from Teknova, Hollister, Calif.) with about 0.2% (v/v) Pluronic L-64 (BASF, Mount Olive, N.J.). About fifteen microliters of each dilution was pipetted into each loading chamber of the Fastman sample processing device. The lysostaphin tablet was dissolved by varying the motor speed on the FastMan unit, which rotates the Fastman processing device. Substantially complete dissolution of the lysostaphin tablet and lysing of the S. aureus took about ten minutes.

Approximately 15 μL of a 20 mg/mL solution of Proteinase K (QIAGEN, Valencia, Calif.) was then introduced into the Fastman sample processing device, and the solution was mixed and incubated on the sample processing device for about 10 minutes at about 65° C. and then for about 10 minutes at about 90° C. After incubation, the solutions were extracted using a 100-4 pipette and transferred into a clean 0.6-mL microfuge tube. About 60 μL of TE Buffer was added to each tube.

A parallel experiment was run for solution form controls: about 15 μL of each dilution of MSSA was pipetted into three separate 0.6-mL tubes. 60 μL of a 250 ng/μL solution of lysostaphin was added to each tube and the solution was mixed by pipetting up and down. After an approximate 10 minute incubation at room temperature, about 15 μL of a 20 mg/mL solution of proteinase K was added to each tube, and the solution was mixed by pipetting up and down. The tubes were then incubated for about 10 minutes in a water bath set at about 65° C. followed by a second approximately 10 minute water bath incubation at about 90° C.

Next, about 25 μL of each solution (the three dilutions from the Fastman sample processing device and the three dilutions from the solution form controls) were pipetted into six new 0.6-mL microfuge tubes. To each of these tubes was added a 25 μL solution of the components shown in Table 3. RT-PCR assays were run on quadruplicates of three dilution points of 3,000, 300, and 30 cfu equivalents.

TABLE 3
Solutions added to solutions

| Reagent | Volume (μL) |
|---|---|
| LightCycler ® DNA Master HybProbe (Roche, | 32 |
| Indianapolis, IN) | |
| 10 μM Forward-SAfemA (651-672) | 16 |
| 10 μM Reverse-SAfemA (768-792) | 16 |
| 5 μM Probe-SAfemA (678-706) | 32 |
| 25 mM MgCl2 | 64 |

Each solution was mixed by pipetting up and down. Then approximately 10 μL was transferred to a LightCycler capillary (available from Roche, Indianapolis, Ind.). PCR was commenced and data was collected on the Light Cycler 2.0 Real-Time PCR System using the time and temperature parameters specified in Table 4. The results are shown in Table 5.

TABLE 4
LightCycler Thermocycling Conditions

| | Temperature(s) Within | |
|---|---|---|
| Number Of Cycles | Each Cycle (° C.) | Time (seconds)* |
| 1 | 95 | 30 |
| 45 | 95 | 0 |
| | 65 | 25* |

*Data was collected at this point in the cycle during each of the 45 cycles.

TABLE 5
Tabulated Ct Values Comparing Tabletted
Lysostaphin With Wet Controls.

| Enzyme | DNA Sample | | | | |
|---|---|---|---|---|---|
| Master Mix | (cfu equivalent) | Ct Value | | | |
| Tabletted | 3000 | 26.13 | 25.79 | 25.78 | 26.02 |
| Lysostaphin | 300 | 29.93 | 29.70 | 29.70 | 30.05 |
| | 30 | 31.61 | 33.51 | 32.80 | 31.53 |
| Solution | 3000 | 23.36 | 23.50 | 23.40 | 23.53 |
| Form | 300 | 27.73 | 27.95 | 27.82 | 28.05 |
| Controls | 30 | 30.40 | 30.19 | 30.90 | 30.13 |

Ct values are shown for replicates run under each condition.

The data shown in Table 5 demonstrates that the lysostaphin tablets solubilized on the microfluidic device showed substantially equivalent Cts and Ct variability at all three concentrations of cells to the solution form control, which included the lysostaphin in a liquid form. Such data demonstrates the feasibility of use of tablets, such as microtablets, containing reagent within a microfluidic device.

### Example 5

As a first example demonstrating the tabletting of a reagent comprising a active component and a reconstitution buffer, it is believed that the reverse transcriptase and RNA polymerase described in U.S. Pat. No. 5,556,771 to Shen et al., entitled, “STABILIZED COMPOSITIONS OF REVERSE TRANSCRIPTASE AND RNA POLYMERASE FOR NUCLEIC ACID AMPLIFICATION,” may be tabletted by substituting a substantially solid reconstitution buffer for the liquid reconstitution buffer described therein. U.S. Pat. No. 5,556,771 to Shen et al. is incorporated herein by reference in its entirety.

U.S. Pat. No. 5,556,771 to Shen et al. describes the lyophilization of reverse transcriptase and RNA polymerase. It is believed that the lyophilized enzyme preparations described in U.S. Pat. No. 5,556,771 to Shen et al. may be tabletted using sorbitol as a matrix material and a suitable lubricant. For example, it is believed the tablet components may be prepared by triturating the lyophilized enzyme using a mortar and pestle and separately triturating sorbitol and l-leucine. It is believed the tablet components may be further prepared by sieving all three components using an 80 mesh sieve. In one type of tablet, it is believed the sorbitol and lyophilized enzyme powders may be mixed together in a 4/1 ratio by weight, where the powders may be well-mixed via a vortexer for about two minutes. Leucine may be added to this mix in a 1/20 leucine/powder mix ratio, and the mixture including the sorbitol, leucine, and lyophilized enzyme powder may be vortexed again for 30 seconds to ensure complete mixing. This formulation may then be formed into one or more tablets, e.g., by compressing the powdered mixture together via a tablet press. Sorbitol can be alternatively added to the lyophilizate and triturated.

As described in U.S. Pat. No. 5,556,771 to Shen et al., the lyophilized enzyme may require a reconstitution buffer prior to use in an assay. However, U.S. Pat. No. 5,556,771 to Shen et al. describes a liquid reconstitution buffer (0.01% (v/v) TRITON® X-100, 41.6 mM MgCl₂, 1 mM ZnC₂H₃O₂, 10% (v/v) glycerol, 0.3% (v/v) ethanol, 0.02% (w/v) methyl paraben, and 0.01% (w/v) propyl paraben). As previously described, a liquid reconstitution buffer may not be suitable for tabletting. Accordingly, in accordance with the first example, it is believed that the lyophilized enzyme described in U.S. Pat. No. 5,556,771 to Shen et al. may be combined with a solid reconstitution buffer prior to tabletting. It is believed that the reconstitution buffer described in U.S. Pat. No. 5,556,771 to Shen et al. (i.e., Triton X-100) may be suitable replaced by Triton X-405, which is a solid surfactant. In addition, it is believed that the glycerol present in the liquid reconstitution buffer described in U.S. Pat. No. 5,556,771 to Shen et al. can be substituted by sorbitol, which is a solid polyol sugar.

It is believed that tabletting the lyophilized enzyme described in U.S. Pat. No. 5,556,771 with a reconstitution buffer comprising Triton X-405 and sorbitol may provide similar results in the nucleic acid amplification experiments described in U.S. Pat. No. 5,556,771. That is, it is believed that a reconstitution buffer comprising Triton X-405 and sorbitol, as well as tabletting of the lyophilized enzymes described in U.S. Pat. No. 5,556,771 may substantially maintain the enzymatic activities of the reverse transcriptase (RNA-directed DNA polymerase, DNA-directed DNA polymerase and RNAse H).

### Example 6

As a second example demonstrating the tabletting of a reagent comprising a active component and a reconstitution buffer, it is believed that the amplification reagent described in U.S. Pat. No. 5,556,771 to Shen et al. may be tabletted along with the reverse transcriptase and RNA polymerase enzymes by substituting a substantially solid reconstitution buffer for the liquid reconstitution buffer described therein. U.S. Pat. No. 5,556,771 to Shen et al. describes an amplification reagent containing 10.0 mM spermidine, 250 mM imidazole/150 mM glutamic acid (pH 6.8), 99 mM NALC, 12.5% (w/v) PVP, 12.5 mM each of rCTP and rUTP, 31.2 mM each of rATP and rGTP, and 10.0 mM each of dCTP, dGTP, dATP and dTTP (6:2 volume ratio). It is believed that the aforementioned amplification reagent may be prepared for tabletting by lyophilizing the reagent with a substantially solid reconstitution buffer, such as about 1% to about 15% Triton X-405. It is believed that the resulting powder may be tabletted, along with sorbitol and a suitable lubricant. It is believed that sorbitol can be alternatively added to the lyophilizate. In some cases, the Triton X-405 nonionic surfactant may be the lubricant.

It is believed that the tabletted amplification reagent, reverse transcriptase and RNA polymerase enzymes, and substantially solid nonionic reconstitution buffer may be reconstituted by deionized water, and achieve substantially the same results in a reaction as the amplification reagent enzyme preparation described in the Examples of U.S. Pat. No. 5,556,771 to Shen et al.

### Example 7

As a third example demonstrating the tabletting of a reagent comprising a active component and a reconstitution buffer, it is believed that the chemiluminescence reagent described in U.S. Pat. No. 5,556,771 to Shen et al. may be tabletted with a substantially solid reconstitution buffer. According to U.S. Pat. No. 5,556,771 to Shen et al., the reagents used in chemiluminescence in wet chemistry stage are 100 μl of a solution of 10 mM lithium succinate (pH 5.0), 2% (w/v) lithium lauryl sulfate, 1 mM mercaptoethanesulfonic acid, 0.3% (w/v) PVP-40, 230 mM LiOH, 1.2M LiCl, 20 mM EGTA, 20 mM EDTA, 100 mM succinic acid (pH 4.7) and 15 mM 2,2′-dipyridyl disulfide containing approximately 75 femtomoles (fmol) of an acridinium ester-labeled oligonucleotide probe ((+) sense) designed to be complementary to the amplified RNA amplicons.

It is believed that this chemiluminescence reagent described in U.S. Pat. No. 5,556,771 to Shen et al. may be tabletted (e.g., compressed via a tablet press) along with sorbitol as a matrix material and a suitable lubricant. Sorbitol can be alternatively added to the lyophilizate. Prior to use in a reaction, the tablets may be reconstituted by ultrapure water. It is believed that the reconstituted tablets may provide substantially similar amplification results (detected via chemiluminescence) as those described in U.S. Pat. No. 5,556,771 to Shen et al. with respect to a lyophilized reagent that was not tabletted.

### Example 8

As a fourth example demonstrating the tabletting of a reagent comprising a active component and a reconstitution buffer, it is believed that the molecular torches for detecting amplified RNA transcripts described in U.S. Pat. No. 6,835,542 to Becker et al., entitled, “MOLECULAR TORCHES,” may be tabletted with a lubricant and sorbitol as a matrix material. U.S. Pat. No. 6,835,542 to Becker et al. is incorporated herein by reference in its entirety. U.S. Pat. No. 6,835,542 to Becker et al. describes a molecular torch that is designed to provide favorable kinetic and thermodynamic components in an assay to detect the presence of a target nucleic acid sequence.

It is believed that the molecular torch described in U.S. Pat. No. 6,835,542 to Becker et al. may be prepared for tabletting by lyophilizing the molecular torch. In addition, it is believed that sorbitol may be added to the lyophilized molecular torch. The lyophilized molecular torch may be formed into a tablet, along with the sorbitol and lubricant. After the tablet is disposed within a processing device, it is believed that the tablet may be reconstituted by ultrapure water prior to performing an assay with the molecular torches within the tablet. It is believed that the reconstituted tablet may provide substantially similar results in detecting a target nucleic acid sequence as those results described in U.S. Pat. No. 6,835,542 to Becker et al.

Various embodiments of the invention have been described. These and other embodiments are within the scope of the following claims.

1. A method comprising:
selecting at least one reagent; and
forming a tablet comprising the at least one reagent and at least one matrix material, wherein the tablet is sized to fit within at least one chamber of a microfluidic processing device.

2. The method of claim 1, wherein forming the tablet comprises compressing the at least one reagent and the at least one matrix material to define the tablet.

3. The method of claim 1, wherein the tablet further comprises a lubricant material.

4. The method of claim 1, wherein forming the tablet comprises forming the tablet comprising a substantially uniform distribution of the at least one reagent and the at least one matrix material.

5. The method of claim 1, further comprising lyophilizing the at least one reagent and the at least one matrix material prior to forming the tablet.

6. The method of claim 1, wherein the at least one matrix material comprises an insoluble material, the method further comprising spraying the at least one reagent onto the insoluble material and dehydrating the insoluble material prior to forming the tablet.

7. The method of claim 1, further comprising dry mixing the at least one reagent and the at least one matrix material prior to forming the tablet.

8. The method of claim 1, wherein the at least one reagent is configured to be used in at least one of a step of sample preparation, a step of nucleic acid amplification, a step of detection in a process for detecting or assaying a nucleic acid, or a step of detection in a process for detecting or assaying a amino acid.

9. The method of claim 1, where the at least one reagent comprises lysostaphin.

10. The method of claim 1, wherein forming the tablet comprises forming the tablet in an environment comprising a relative humidity of about 1% to about 30%.

11. The method of claim 1, wherein the tablet is a microtablet with a greatest dimension in a range of about 0.5 millimeters to about 5 millimeters.

12. A method comprising:
introducing an analyte into a microfluidic sample processing device; and
at least partially dissolving a tablet in a chamber of the microfluidic device, wherein the tablet comprises a reagent and a matrix material and is configured to fit within the chamber of the microfluidic processing device.

13. The method of claim 12, wherein the matrix material comprises a solubility of about 0 grams per 100 grams of water to about 400 grams per 100 grams of water.

14. The method of claim 13, wherein the tablet substantially dissolves in the chamber within about 30 seconds to about 300 seconds from an introduction of a fluid into the chamber.

15. The method of claim 12, further comprising processing the analyte with the reagent, wherein processing the sample comprises at least one of preparing the sample, nucleic acid amplification, detecting or assaying a nucleic acid or detecting or assaying an amino acid.

16. The method of claim 1, wherein the reagent comprises at least one of a lysis reagent, a protein-digesting reagent, a nucleic acid amplifying enzyme, an oligonucleotide, a probe, nucleotide triphosphates, a buffer, a salt, a surfactant, a dye, a nucleic acid control, a reducing agent, dimethyl sulfoxide (DMSO), ethylenediaminetetraacetic acid (EDTA), ethylene glycol-bis(2-aminoethylether)-N,N,N′,N′-tetraacetic acid (EGTA), microspheres capable of binding a nucleic acid or a combination thereof.

17. The method of claim 1, wherein the matrix material comprises at least one of a water soluble polymer, a carbohydrate and a combination thereof.

18. The method of claim 1, wherein the tablet includes about 1 percent to about 95 percent by tablet weight of the reagent.

19. The method of claim 1, wherein the at least one reagent includes a first reagent and a second reagent; wherein the first reagent comprises an active component, wherein the active component requires a reconstitution buffer prior to use in a chemical reaction; and wherein the second reagent comprises a substantially solid reconstitution buffer.

20. An assembly comprising:
a microfluidic processing device comprising:
an input chamber; and
a process chamber fluidically coupled to the input chamber; and
a tablet comprising a reagent and a matrix material, wherein the tablet is configured to fit within the process chamber of the microfluidic processing device.

21. The assembly of claim 20, wherein the at least one reagent comprises at least one of a lysis reagent, a protein-digesting reagent, a nucleic acid amplifying enzyme, an oligonucleotide, a probe, nucleotide triphosphates, a buffer, a salt, a surfactant, a dye, a nucleic acid control, a reducing agent, dimethyl sulfoxide (DMSO), ethylenediaminetetraacetic acid (EDTA), ethylene glycol-bis(2-aminoethylether)-N,N,N′,N′-tetraacetic acid (EGTA), microspheres capable of binding a nucleic acid or a combination thereof.

22. The assembly of claim 20, wherein the matrix material comprises at least one of a water soluble polymer, a carbohydrate and a combination thereof.

23. The assembly of claim 20, wherein the tablet includes about 1 percent to about 95 percent by tablet weight of the reagent.

24. A method comprising:
selecting a active component, wherein the active component requires a reconstitution buffer prior to use in a chemical reaction;
selecting a substantially solid reconstitution buffer; and
forming a tablet comprising the active component and the substantially solid reconstitution buffer, wherein the tablet is sized to fit within at least one chamber of a microfluidic processing device.

25. The method of claim 24, wherein the solid reconstitution buffer comprises a nonionic solid surfactant.

26-29. (canceled)
